SEQUENCE LISTING (52 sequences)

SEQ ID NO: 1 - protein, 7 aa, synthetic construct
  REGION 1..7: note = Synthetic
FTWDHYI

SEQ ID NO: 2 - protein, 7 aa, synthetic construct
  REGION 1..7: note = Synthetic
ENGDAWN

SEQ ID NO: 3 - protein, 18 aa, synthetic construct
  REGION 1..18: note = Synthetic
IGFDVPSGRSWQGSHFWM

SEQ ID NO: 4 - protein, 7 aa, synthetic construct
  REGION 1..7: note = Synthetic
TTSRWES

SEQ ID NO: 5 - protein, 7 aa, synthetic construct
  REGION 1..7: note = Synthetic
WQNNSVP

SEQ ID NO: 6 - protein, 9 aa, synthetic construct
  REGION 1..9: note = Synthetic
AQHNFLGHR

SEQ ID NO: 7-8 - skipped (2 entries)

SEQ ID NO: 9 - protein, 128 aa, synthetic construct
  REGION 1..128: note = Synthetic
EVQLQASGGGFVQPGGSLRLSCAASGFTWDHYIMGWFRQAPGKEREFVSAISENGDAWNYYADSVKGRFTISRDNSKNTVYLQMNSLRAEDTATYYCAIGFDVPSGRSWQGSHFWMYWGQGTQVTVSS

SEQ ID NO: 10 - protein, 119 aa, synthetic construct
  REGION 1..119: note = Synthetic
EVQLQASGGGFVQPGGSLRLSCAASGTTSRWESMGWFRQAPGKEREFVSAISWQNNSVPYYADSVKGRFTISRDNSKNTVYLQMNSLRAEDTATYYCAAQHNFLGHRYWGQGTQVTVSS

SEQ ID NO: 11 - protein, 502 aa, synthetic construct
  REGION 1..502: note = Synthetic
  VARIANT 1: note = optionally absent
MSRDPLPFFPPLYLGGPEITTENCEREPIHIPGSIQPHGALLTADGHSGEVLQMSLNAATFLGQEPTVLRGQTLAALLPEQWPALQAALPPGCPDALQYRATLDWPAAGHLSLTVHRVGELLILEFEPTEAWDSTGPHALRNAMFALESAPNLRALAEVATQTVRELTGFDRVMLYKFAPDATGEVIAEARREGLHAFLGHRFPASDIPAQARALYTRHLLRLTADTRAAAVPLDPVLNPQTNAPTPLGGAVLRATSPMHMQYLRNMGVGSSLSVSVVVGGQLWGLIACHHQTPYVLPPDLRTTLEYLGRLLSLQVQVKEAADVAAFRQSLREHHARVALAAAHSLSPHDTLSDPALDLLGLMRAGGLILRFEGRWQTLGEVPPAPAVDALLAWLETQPGALVQTDALGQLWPAGADLAPSAAGLLAISVGEGWSECLVWLRPELRLEVAWGGATPDQAKDDLGPRHSFDTYLEEKRGYAEPWHPGEIEEAQDLRDTLTGAL

SEQ ID NO: 12 - protein, 7 aa, synthetic construct
  REGION 1..7: note = Synthetic
DTSYLYS

SEQ ID NO: 13 - protein, 7 aa, synthetic construct
  REGION 1..7: note = Synthetic
WWWNLTQ

SEQ ID NO: 14 - protein, 15 aa, synthetic construct
  REGION 1..15: note = Synthetic
WSIYFPPGNDYNGYH

SEQ ID NO: 15 - protein, 7 aa, synthetic construct
  REGION 1..7: note = Synthetic
FFSNWSD

SEQ ID NO: 16 - protein, 7 aa, synthetic construct
  REGION 1..7: note = Synthetic
FWADGTE

SEQ ID NO: 17 - protein, 12 aa, synthetic construct
  REGION 1..12: note = Synthetic
WYGPVNGFYMFD

SEQ ID NO: 18 - protein, 7 aa, synthetic construct
  REGION 1..7: note = Synthetic
STSDFES

SEQ ID NO: 19 - protein, 7 aa, synthetic construct
  REGION 1..7: note = Synthetic
SWFTNPP

SEQ ID NO: 20 - protein, 9 aa, synthetic construct
  REGION 1..9: note = Synthetic
HRSIWYHPT

SEQ ID NO: 21 - DNA, 1659 bp, synthetic construct
  misc_feature 1..1659: note = Synthetic
  source 1..1659: mol_type = other DNA
atgagtcgtgaccctttgccattctttcctcctctttatctgggtggacccgagattacaacagaaaactgcgaacgcgaaccaattcacatcccgggatctattcaaccacacggtgcattgctgacggcagacggacattccggagaggttttacagatgtcgcttaacgcagcaacgtttctgggacaagagcctacggttttgcgcggccagacgttagcggctctgttgccagagcaatggccggccttacaggcggcattgcctccagggtgccccgatgcattgcaataccgcgcgacactggattggccggcggcaggacatctttctctgacagtccaccgcgtgggcgagctgttgatcctggagtttgaacctacggaggcctgggactcgactggcccgcacgcgttacgcaatgcgatgttcgctcttgaatcagcgccaaacttgcgcgcgttagctgaagtggccacacaaaccgtacgcgagcttacaggctttgaccgcgtgatgttatacaaattcgcacccgatgcgacaggcgaggtaatcgccgaagcccgccgcgaggggttgcatgcctttcttggccatcgttttccggcctcagatattcccgcccaagcgcgcgccctttacactcgccatctgcttcgtttgactgcggacacgcgcgcggcggccgttcccttagacccagtacttaatcctcagactaacgctcctacccccttagggggggcagtgctgcgtgcgacgtcgcctatgcacatgcagtaccttcgcaatatgggcgtcggctcctctttaagtgtatcagtggtagttggggggcagttatggggtctgattgcgtgccatcatcagaccccctatgttttgccaccagaccttcgtactactcttgaatacttggggcgtttattaagccttcaggtgcaagtcaaggaagccgcggacgttgctgcattccgtcagtcacttcgcgaacaccatgcgcgcgtcgccttagcggcagcgcattccctgtcgccgcacgatactctttccgaccctgcacttgatcttctgggtctgatgcgtgctgggggcttaatcctgcgttttgaaggtcgttggcagacgttaggagaagtcccgcccgctcccgcagtcgatgcactgcttgcatggcttgaaacccaaccaggggcgcttgttcagactgatgcattggggcagttgtggccggcgggggctgatttggctccctcagccgcgggtctgcttgccatttcagtaggggagggatggagtgagtgcttggtttggttacgtcccgaactgcgccttgaggttgcgtggggtggagcaactccagaccaggccaaggacgacctgggccctcgtcacagtttcgatacttacttagaagagaagcgtgggtatgcagaaccctggcatcccggagagattgaggaagctcaggatttgcgcgacactcttactggcgcattaaagcttggtggcggtagcgagaatttgtattttcagggtggcggtggcagtagcttatccaccccgccgaccccgagcactcctcctaccggtctgaacgacatcttcgaggctcagaaaatcgaatggcacgaacatcatcaccaccatcac

SEQ ID NO: 22 - DNA, 24 bp, synthetic construct
  misc_feature 1..24: note = Synthetic
  source 1..24: mol_type = other DNA
gaattccatcatcaccatcaccat

SEQ ID NO: 23 - DNA, 417 bp, synthetic construct
  misc_feature 1..417: note = Synthetic
  source 1..417: mol_type = other DNA
gaagttcagctgcaggcaagcggtggtggttttgttcagcctggtggtagcctgcgtctgagctgtgcagccagcggttttacctgggatcattacatcatgggctggtttcgccaggcaccgggtaaagaacgtgaatttgttagcgcaatcagcgaaaatggtgatgcatggaattattatgccgatagcgtgaaaggtcgctttaccattagccgtgataatagcaaaaataccgtttacctgcagatgaatagtctgcgtgcagaagataccgcaacctattattgtgcaatcggttttgatgttccatctggtcgttcttggcagggttctcatttttggatgtattggggtcagggcacccaggttaccgttagcagcagcccgggaggccaacaccatcaccaccatcat

SEQ ID NO: 24 - DNA, 126 bp, synthetic construct
  misc_feature 1..126: note = Synthetic
  source 1..126: mol_type = other DNA
agcccgggaggccaaagcttatccaccccgagtgtagatctcggtggtcgccgtatcattggtctgaacgacatcttcgaggctcagaaaatcgaatggcacgaacatcatcaccaccatcactct

SEQ ID NO: 25 - DNA, 390 bp, synthetic construct
  misc_feature 1..390: note = Synthetic
  source 1..390: mol_type = other DNA
gaagttcagctgcaggcaagcggtggtggttttgttcagcctggtggtagcctgcgtctgagctgtgcagccagcggtaccacctctcgttgggaatctatgggctggtttcgccaggcaccgggtaaagaacgtgaatttgttagcgcaatcagctggcagaataattctgttccatattatgccgatagcgtgaaaggtcgctttaccattagccgtgataatagcaaaaataccgtttacctgcagatgaatagtctgcgtgcagaagataccgcaacctattattgtgcagcacagcataactttctgggtcatcgttattggggtcagggcacccaggttaccgttagcagcagcccgggaggccaacaccatcaccaccatcat

SEQ ID NO: 26 - DNA, 126 bp, synthetic construct; identical to SEQ ID NO: 24
  misc_feature 1..126: note = Synthetic
  source 1..126: mol_type = other DNA

SEQ ID NO: 27 - DNA, 522 bp, synthetic construct
  misc_feature 1..522: note = Synthetic
  source 1..522: mol_type = other DNA
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaatttgtaatacgactcactatagggcgagccgccatcatggaggagcagaagctgatctcagaggaggacctgcat

SEQ ID NO: 28 - DNA, 507 bp, synthetic construct
  misc_feature 1..507: note = Synthetic
  source 1..507: mol_type = other DNA
atggataaagcggaattaattcccgagcctccaaaaaagaagagaaaggtcgaattgggtaccgccgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagatctttaatacgactcactatagggcgagcgccgccatggagtacccatacgacgtaccagattacgctcatatgggaggcggttccggtggcggttct

SEQ ID NO: 29 - DNA, 474 bp, synthetic construct
  misc_feature 1..474: note = Synthetic
  source 1..474: mol_type = other DNA
atctttaatacgactcactatagggcgagcgccgccatggagtacccatacgacgtaccagattacgctcatatgggaggcggttccggtggcggttctgaagttcagctgcaggcaagcggtggtggttttgttcagcctggtggtagcctgcgtctgagctgtgcagccagcggtgatacctcttacctgtactctatgggctggtttcgccaggcaccgggtaaagaacgtgaatttgttagcgcaatcagctggtggtggaatctgactcagtattatgccgatagcgtgaaaggtcgctttaccattagccgtgataatagcaaaaataccgtttacctgcagatgaatagtctgcgtgcagaagataccgcaacctattattgtgcatggtctatctactttccaccaggtaacgattacaacggttaccattattggggtcagggcacccaggttaccgttagcagc

SEQ ID NO: 30 - DNA, 465 bp, synthetic construct
  misc_feature 1..465: note = Synthetic
  source 1..465: mol_type = other DNA
atctttaatacgactcactatagggcgagcgccgccatggagtacccatacgacgtaccagattacgctcatatgggaggcggttccggtggcggttctgaagttcagctgcaggcaagcggtggtggttttgttcagcctggtggtagcctgcgtctgagctgtgcagccagcggttttttttctaactggtctgatatgggctggtttcgccaggcaccgggtaaagaacgtgaatttgttagcgcaatcagcttttgggcagatggtactgaatattatgccgatagcgtgaaaggtcgctttaccattagccgtgataatagcaaaaataccgtttacctgcagatgaatagtctgcgtgcagaagataccgcaacctattattgtgcatggtacggtccagttaacggtttttacatgtttgattattggggtcagggcacccaggttaccgttagcagc

SEQ ID NO: 31 - DNA, 456 bp, synthetic construct
  misc_feature 1..456: note = Synthetic
  source 1..456: mol_type = other DNA
atctttaatacgactcactatagggcgagcgccgccatggagtacccatacgacgtaccagattacgctcatatgggaggcggttccggtggcggttctgaagttcagctgcaggcaagcggtggtggttttgttcagcctggtggtagcctgcgtctgagctgtgcagccagcggttctacctctgattttgaatctatgggctggtttcgccaggcaccgggtaaagaacgtgaatttgttagcgcaatcagctcttggtttactaatccaccatattatgccgatagcgtgaaaggtcgctttaccattagccgtgataatagcaaaaataccgtttacctgcagatgaatagtctgcgtgcagaagataccgcaacctattattgtgcacatcgttctatctggtaccatccaacctattggggtcagggcacccaggttaccgttagcagc

SEQ ID NO: 32 - DNA, 99 bp, synthetic construct
  misc_feature 1..99: note = Synthetic
  source 1..99: mol_type = other DNA
atctttaatacgactcactatagggcgagcgccgccatggagtacccatacgacgtaccagattacgctcatatgggaggcggttccggtggcggttct

SEQ ID NO: 33 - DNA, 984 bp, synthetic construct
  misc_feature 1..984: note = Synthetic
  source 1..984: mol_type = other DNA
atgggatcccccaagaagaagcgcaaggtggaagctagcgcttccccgaagaaaaagcggaaagtcgaggcctccgcatctccaaaaaaaaaaagcaaggttgaagcatctggatccggtaccggaggaagtggcagctctggcggcagtggagggtctggtggcagcggaagcggcggcggtggcagtcagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 34 - DNA, 78 bp, synthetic construct
  misc_feature 1..78: note = Synthetic
  source 1..78: mol_type = other DNA
ggatccggtaccggaggaagtggcagctctggcggcagtggagggtctggtggcagcggaagcggcggcggtggcagt

SEQ ID NO: 35 - DNA, 78 bp, synthetic construct; identical to SEQ ID NO: 34
  misc_feature 1..78: note = Synthetic
  source 1..78: mol_type = other DNA

SEQ ID NO: 36 - DNA, 78 bp, synthetic construct; identical to SEQ ID NO: 34
  misc_feature 1..78: note = Synthetic
  source 1..78: mol_type = other DNA

SEQ ID NO: 37 - DNA, 219 bp, synthetic construct
  misc_feature 1..219: note = Synthetic
  source 1..219: mol_type = other DNA
atgcccaagaagaagcgcaaggtggaagctagcgcttccccgaagaaaaagcggaaagtcgaggcctccgcatctccaaaaaaaaaaagcaaggttgaagcatctggatccggaggcggttccggtggcggttctggtaccggaggaagtggcagctctggcggcagtggagggtctggtggcagcggaggaggaagtggcagctctggcggcagtgga

SEQ ID NO: 38 - DNA, 2274 bp, synthetic construct
  misc_feature 1..2274: note = Synthetic
  source 1..2274: mol_type = other DNA
ccggaatttgtaatacgactcactatagggcgagccgccatcatggaggagcagaagctgatctcagaggaggacctgcatgtggcaggtcatgcctctggcagccccgcattcgggaccgccgatctttcgaattgcgaacgtgaagagatccacctcgccggctcgatccagccgcatggcgcgcttctggtcgtcagcgagccggatcatcgcatcatccaggccagcgccaacgccgcggaatttctgaatctcggaagcgtgctcggcgttccgctcgccgagatcgacggcgatctgttgatcaagatcctgccgcatctcgatcccaccgccgaaggcatgccggtcgcggtgcgctgccggatcggcaatccctccacggagtacgacggtctgatgcatcggcctccggaaggcgggctgatcatcgagctcgaacgtgccggcccgccgatcgatctgtccggcacgctggcgccggcgctggagcggatccgcacggcgggctcgctgcgcgcgctgtgcgatgacaccgcgctgctgtttcagcagtgcaccggctacgaccgggtgatggtgtatcgcttcgacgagcagggccacggcgaagtgttctccgagcgccacgtgcccgggctcgaatcctatttcggcaaccgctatccgtcgtcggacattccgcagatggcgcggcggctgtacgagcggcagcgcgtccgcgtgctggtcgacgtcagctatcagccggtgccgctggagccgcggctgtcgccgctgaccgggcgcgatctcgacatgtcgggctgcttcctgcgctcgatgtcgccgatccatctgcagtacctgaagaacatgggcgtgcgcgccaccctggtggtgtcgctggtggtcggcggcaagctgtggggcctggttgcctgtcaccattatctgccgcgcttcatccatttcgagctgcgggcgatctgcgaactgctcgccgaagcgatcgcgacgcggatcaccgcgcttgagagcttcgcgcagagccagtcggagctgttcgtgcagcggctcgaacagcgcatgatcgaagcgatcacccgtgaaggcgattggcgcgcagcgattttcgacaccagccaatcgatcctgcagccgctgcacgccgacggttgcgcgctggtgtacgaagaccagatcaggaccatcggtgacgtaccttccacgcaggatgttcgcgagatcgccgggtggctcgatcgccagccacgtgcggcggtgacctcgaccgcgtcgctcggtctcgacgtgccggagctcgcgcatctgacgcggatggcgagcggcgtggtcgcggcgccgatttcggatcatcgcggcgagtttctgatgtggttccgccccgagcgcgtccacaccgttacctggggcggcgatccgaagaagccgttcacgatgggcgatacaccggcggatctgtcgccgcggcgctccttcgccaaatggcatcaggttgtcgaaggcacgtccgatccgtggacggccgccgatctcgccgcggctcgcaccatcggtcagaccgtcgccgacatcgtgctgcaattccgcgcggtgcggacactgatcgcccgcgaacagtacgaacagttttcgtcccaggtgcacgcttcgatgcagccggtgctgatcaccgacgccgaaggccgcatcctgctgatgaacgactcgttccgcgacatgttgccggcggggtcgccatccgccgtccatctcgacgatctcgccgggttcttcgtcgaatcgaacgatttcctgcgcaacgtcgccgaactgatcgatcacggccgcgggtggcgcggcgaagttctgctgcgcggcgcaggtaatcgcccgttgccgctggcagtgcgcgccgatccggtgacgcgcacggaggaccagtcgctcggcttcgtgctgatcttcagcgacgctaccgatcgtcgcaccgcagatgccgcacgcacgcgtttccaggaaggcattcttgccagcgcacgtcccggcgtgcggctcgactccaagtccgacctcttgcacgagaagctgctgtccgcgctggtcgagaacgcgcagcttgccgcattggaaattacttacggcgtcgagaccggacgcatcgccgagctgctcgaaggcgttcgccagtcgatgctgcgcaccgccgaagtgctcggccatctggtgcagcacgcggcgcgcacggccggcagcgacagctcgagcaatggctcgcagaacaagaag

SEQ ID NO: 39 - DNA, 1611 bp, synthetic construct
  misc_feature 1..1611: note = Synthetic
  source 1..1611: mol_type = other DNA
atgcccaagaagaagcgcaaggtggaagctagcgcttccccgaagaaaaagcggaaagtcgaggcctccgcatctccaaaaaaaaaaagcaaggttgaagcatctggatccggaggcggttccggtggcggttctgtggcgtcaaagtccgttcatgccgacatcacccttctgctcgatatggagggtgtgattcgcgaagccaccctgtctccgacgatggcggccgagagcgtggacggttggctggggcgtcgctggagcgacatcgccggcgccgaaggcggcgacaaggttcgccgcatggtcgaagacgcccgccgcagcggcatctcggctttccgccagatcaatcagcctttcccgagcggcgtcgaaatcccgatcgaattcaccacgatgctgctgggcgaccgcaccggcatgatcgcggtcggcaagaacatgcaggcggtcaccgagctgcattcccggctgatcgctgcgcagcaggcgatggagcgcgactattggcggttgcgtgaattggagactcgctaccgcctggtgttcgacgctgccgccgatgcggtgatgatcgtctccgccggcgacatgcgcatcgtcgaagccaaccgggcggcggtgaatgcgatcagccgcgtcgagcgcggcaatgacgaccttgcggggcgtgatttcctcgccgaagtggcggctgccgatcgcgatgcggtgcgcgacatgctggcccaggtgcgtcagcgcggcaccgcactcagcgtcctcgttcatctcggccgttacgaccgcgcctggatgctgcgcggttcgctgatgtcgtccgagcgtcgtcaggttttcctgctgcacttcaccccggtgaccacgactcccgcgatcgacgacgtcgacgatgatgccgtgctgcgcgggctgatcgatcgcattcccgacgggttcgtcgcactggattcggaaggcgtcgttcgtcacgccaaccaggcgtttctcgatctggtccagatcggctccaagcctgcggcggtcggacgatcgctgggcgtctggatgggtcgtccgggcgccgatctgtccagcttgctgacgctgctgcggcgctacaagacggtgcggctgttccaaacgacgatccgcggcgagctcggcaccgagactgaagtcgaggtctcggccgtcgacggcgaggacgaccaatacatcggcgttctgatgcgcaatgtcgcgcgacgcctcgacgctgcggacgaccacgatgccttgcgtcaggcgctcggcccgatcagcaagcagctcgggcgatcctcgctgcgcaagctggtgaagaacgccgtgagcattgtcgagcagcactacgtgaaggaagcgctgttgcgatccaagggcaatcgcacggcaactgccgaactgctcggattgagccggcagagcctttatgcaaaactcaactcctacggcttcgacgacaaaggtgtcgttgcttctgctgccgacggtgcagagggcgcctcagacgacgcagaggatggtaccggaggaagtggcagctctggcggcagtggagggtctggtggcagcggaggaggaagtggcagctctggcggcagtgga

SEQ ID NO: 40 - DNA, 672 bp, synthetic construct
  misc_feature 1..672: note = Synthetic
  source 1..672: mol_type = other DNA
atgcccaagaagaagcgcaaggtggaagctagcgcttccccgaagaaaaagcggaaagtcgaggcctccgcatctccaaaaaaaaaaagcaaggttgaagcatctggatccggaggcggttccggtggcggttctggcaagaacatgcaggcggtcaccgagctgcattcccggctgatcgctgcgcagcaggcgatggagcgcgactattggcggttgcgtgaattggagactcgctaccgcctggtgttcgacgctgccgccgatgcggtgatgatcgtctccgccggcgacatgcgcatcgtcgaagccaaccgggcggcggtgaatgcgatcagccgcgtcgagcgcggcaatgacgaccttgcggggcgtgatttcctcgccgaagtggcggctgccgatcgcgatgcggtgcgcgacatgctggcccaggtgcgtcagcgcggcaccgcactcagcgtcctcgttcatctcggccgttacgaccgcgcctggatgctgcgcggttcgctgatgtcgtccgagcgtcgtcaggttttcctgctgcacttcaccccggtgaccacgactcccgcgatcgacgacggtaccggaggaagtggcagctctggcggcagtggagggtctggtggcagcggaggaggaagtggcagctctggcggcagtgga

SEQ ID NO: 41 - DNA, 573 bp, synthetic construct
  misc_feature 1..573: note = Synthetic
  source 1..573: mol_type = other DNA
atgggatccgttaacggcggcggtggcagtgacaaagactgcgaaatgaagcgcaccaccctggatagccctctgggcaagctggaactgtctgggtgcgaacagggcctgcaccgtatcatcttcctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctacagccacctggccgccctggccggcaatcccgccgccaccgccgccgtgaaaaccgccctgagcggaaatcccgtgcccattctgatcccctgccaccgggtggtgcagggcgacctggacgtggggggctacgagggcgggctcgccgtgaaagagtggctgctggcccacgagggccacagactgggcaagcctgggctgggt

SEQ ID NO: 42 - DNA, 30 bp, synthetic construct
  misc_feature 1..30: note = Synthetic
  source 1..30: mol_type = other DNA
atgggatccgttaacggcggcggtggcagt

SEQ ID NO: 43 - DNA, 30 bp, synthetic construct; identical to SEQ ID NO: 42
  misc_feature 1..30: note = Synthetic
  source 1..30: mol_type = other DNA

SEQ ID NO: 44 - DNA, 30 bp, synthetic construct; identical to SEQ ID NO: 42
  misc_feature 1..30: note = Synthetic
  source 1..30: mol_type = other DNA

SEQ ID NO: 45 - DNA, 30 bp, synthetic construct; identical to SEQ ID NO: 42
  misc_feature 1..30: note = Synthetic
  source 1..30: mol_type = other DNA

SEQ ID NO: 46 - protein, 26 aa, synthetic construct
  REGION 1..26: note = Synthetic
EVQLQASGGGFVQPGGSLRLSCAASG

SEQ ID NO: 47 - protein, 19 aa, synthetic construct
  REGION 1..19: note = Synthetic
MGWFRQAPGKEREFVSAIS

SEQ ID NO: 48 - protein, 39 aa, synthetic construct
  REGION 1..39: note = Synthetic
YYADSVKGRFTISRDNSKNTVYLQMNSLRAEDTATYYCA

SEQ ID NO: 49 - protein, 12 aa, synthetic construct
  REGION 1..12: note = Synthetic
YWGQGTQVTVSS

SEQ ID NO: 50 - protein, 26 aa, synthetic construct
  REGION 1..26: note = Synthetic
  VARIANT 3: note = X is K, Q, N, R, D, or E
  VARIANT 12: note = X is D, G, E, A, V, L, or I
  VARIANT 13: note = X is L, F, W, G, A, V, L, or I
  VARIANT 22: note = X is K, R, D, E, N, or Q
EVXLQASGGGFXXPGGSLRLSXAASG

SEQ ID NO: 51 - protein, 19 aa, synthetic construct
  REGION 1..19: note = Synthetic
  VARIANT 4: note = X is V, P, G, A, L, I, or M
  VARIANT 7: note = X is T, A, S, G, V, L, or I
  VARIANT 9: note = X is E, G, D, A, V, L, or I
MGWXRQXPXKEREFVSAIS

SEQ ID NO: 52 - protein, 39 aa, synthetic construct
  REGION 1..39: note = Synthetic
  VARIANT 3: note = X is P, A, M, G, V, L, or I
  VARIANT 5: note = X is T or S
  VARIANT 17: note = X is A, S, M, G, V, L, or I
  VARIANT 21: note = X is L, V, G, A, or I
  VARIANT 26: note = X is S, N, Q, or T
  VARIANT 29: note = X is K, R, D, or E
  VARIANT 30: note = X is S, A, T, G, V, L, or I
  VARIANT 35: note = X is M, T, V, L, or I
  VARIANT 39: note = X is V, G, L, I, or A
YYXDXVKGRFTISRDNXKNTXYLQMXSLXXEDTAXYYCX

(19) United States
(12) Patent Application Publication
(10) Pub. No.: US 2026/0285953 A1
(43) Pub. Date: Sep. 24, 2026

(54) Red Light-Controlled Protein Dimerization Systems

(71) Applicant: University of Washington, Seattle, WA (US)

(72) Inventors: Liangcai GU, Seattle, WA (US); Zhimin HUANG, Seattle, WA (US); Xiao ZHANG, Seattle, WA (US)

(21) Appl. No.: 19/698,431

(22) Filed: Jun. 4, 2026

Related U.S. Application Data
(63) Continuation of application No. 17/997,582, filed on Jun. 17, 2021, now Pat. No. 12,679,884.
(60) Provisional application No. 63/041,688, filed on Jun. 19, 2020.

(51) Int. Cl. C07K 16/1267

(52) U.S. Cl. CPC C07K 16/1267; C07K 2317/22; C07K 2317/569; C07K 2317/92; C07K 2317/94

(57) ABSTRACT

Deinococcus radiodurans phytochrome (DrBphP) light form-binding antibodies are disclosed and their use in light-induced dimerization systems.

### FEDERAL FUNDING STATEMENT

This invention was made with government support under Grant No. R35 GM128918, awarded by the National Institutes of Health. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Jun. 2, 2026, having the file name “20-946-WO-US-CON.xml” and is 82,368 bytes in size.

### BACKGROUND

Protein dimerization systems that can be controlled by red light with increased tissue penetration depth are highly needed tool for clinical applications such as cell and gene therapies. However, existing red light-induced dimerization systems are all based on phytochrome photoreceptors and naturally occurring binding partners with complex structures and suboptimal in vivo performance, limiting mammalian applications.

### SUMMARY

In one aspect, the disclosure provides Deinococcus radiodurans bacteriophytochrome (DrBphP) light form-binding antibodies, comprising a set of complementarity-determining regions (CDRs) selected from the group consisting of:
(a) SEQ ID NO:1 (CDR1), SEQ ID NO:2 (CDR2), and SEQ ID NO:3 (CDR3); or
(b) SEQ ID NO:4 (CDR1), SEQ ID NO:5 (CDR2), and SEQ ID NO:6 (CDR3).

In various embodiments, the antibody may be a monoclonal antibody, wherein the antibody is selected from the group consisting of humanized antibody, chimeric antibody, Fab′, F(ab′)₂, Fab, Fv, rIgG, recombinant single chain Fv fragments (scFv), single-domain antibody (nanobody), bivalent or bispecific molecule, diabody, triabody, and tetrabody. In one embodiment, the antibody comprises a single-domain antibody.

In a further embodiment, the antibody comprises an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical along the length of the amino acid sequence of SEQ ID NO:9 or SEQ ID NO:10.

In further aspects, the disclosure provides nucleic acids encoding the antibodies of the disclosure, expression vectors comprising the nucleic acids operatively linked to a control sequence, cells comprising the antibody, nucleic acid, or expression vector of any preceding claim, and kits comprising:
(a) the antibody, nucleic acid, expression vector, and/or cell of any of preceding claim; and
(b) a photosensory module of Deinococcus radiodurans bacteriophytochrome (DrBphP), including but not limited to a polypeptide at least 70%, 75%, 80%, 85%, 90%, 91%, 92%, 93%, 94%, 95%, 96%, 97%, 98%, 99%, or 100% identical to the amino acid sequence of SEQ ID NO: 11, wherein optional residues in parentheses may be present or absent and are not considered when determining the percent identity when absent, a nucleic acid encoding such a photosensory module of DrBphP, an expression vector comprising the nucleic acid encoding such a photosensory module of DrBphP operatively linked to a control sequence, and/or cell comprising the photosensory module of DrBphP, the nucleic acid encoding the photosensory module of DrBphP, and/or the expression vector comprising the nucleic acid encoding a photosensory module of DrBphP operatively linked to a control sequence.

The disclosure further provides methods for use of the antibodies, kits, nucleic acids, expression vectors, or host cell of any embodiment or combination of embodiments disclosed herein for any suitable purpose, including but not limited to use as a light induced dimerization (LID) system for any purpose, including but not limited to control light activated gene expression and spatiotemporal activation of chimeric antigen receptor T (CAR-T) cells, etc.

In another aspect, the disclosure provides methods for making a light induced dimerization (LID) system, comprising
(a) phage display to enrich binders that only bind to the light form of a conformation switcher (i.e.: changes conformation when exposed to light); and
(b) yeast two-hybrid (Y2H) screening of the enriched sub-library to select for in vivo LID activity. In one embodiment, the method comprises
(a) screening a synthetic combinatorial polypeptide library with a light form of a conformation switcher to identify binding polypeptides; and
(b) screening a synthetic combinatorial polypeptide library with a dark form of a conformation switcher (conformation assumed when not exposed to light) to identify dimerization polypeptides in the library that (i) bind to the light form of a conformation switcher, and (ii) do not bind to the dark form of a conformation switcher.

### DESCRIPTION OF THE FIGURES

FIG. 1(a-b). Schematics of LID (a) and the COMBINES-LID method (b).

FIG. 2(a-b). Y2H and single phage ELISA analyses of dimerization binder candidates. (a) Y2H assay with the biliverdin-bound DrBphP photosensory module as a bait and nanobodies as preys. A serial dilution of Y2HGold cells resuspended in 0.9% NaCl were spotted on SD/-Ade/-His/-Leu/-Trp plates and grown under the 654-nm illumination (0.03 mW/cm²) or in the dark. A representative result from three independent experiments is shown on the right. (b) ELISA analysis of nanobody binding specificity and reversibility. Phage-displayed nanobodies were bound to DrBphP immobilized in microtiter plates, which were illuminated with the 654-nm (0.3 mW/cm²) or 775-nm (0.2 mW/cm²) lights during the binding and wash steps. Data represent mean values of 3 measurements; error bars, standard deviation.

FIG. 3(a-b). Nanobody specificity validation in mammalian cells. (a) Schematic of the M2H assay. DrBphP and nanobody genes were inserted into the bait and prey plasmids, respectively. (b) Specificity comparison of LID systems. HEK293T cells were transiently co-transfected with the bait, prey, and GAL4UAS-luciferase reporter plasmids (~0.25 μg each) in a 0.5 mL culture. None, the negative control transfected with only the bait and the luciferase reporter plasmids. Cells were cultured under the illumination (654-nm (0.2 mW/cm²) or 775-nm (0.2 mW/cm²)) or in the dark for 24 hours before measuring luciferase levels. Different from DrBphP, RpBphP1 is required to be converted to the light form by a NIR (e.g., 775-nm) light. Data represent mean values of 3 measurements; error bars, standard deviation.

FIG. 4(a-b). Detection of light-induced DrBphP-nanobody complexes (a) Analytical SEC. ~6 μM (final concentration) DrBphP after the 654-nm (0.2 mW/cm²) or 775-nm (0.8 mW/cm²) illumination for 5 min were incubated with ~5 μM (final concentration) LDB-3 or LDB-14 in the dark. 500 uL mixtures were loaded onto a Superdex™ 200 Increase 10/300 GL column pre-equilibrated with 1×PBS buffer and eluted at 0.75 mL/min at 4° C. Elution volumes of protein standards are marked by triangles. Fractions (500 μL each; marked by dash lines) were collected and concentrated by trichloroacetic acid precipitation for SDS-PAGE analysis (b). Only gel regions showing DrBphP and nanobody bands are shown.

FIG. 5(a-b). ITC thermographs of the nanobody binding. 80 μM LDB-3 (a) and 50 μM LDB-14 (b) were titrated into 10 μM and 5 μM DrBphP, respectively. The light and dark forms were converted by the 654-nm (0.2 mW/cm²) and 775-nm (0.2 mW/cm²) lights, respectively. The raw data (top) and the integration of heats (bottom) for each titration are shown.

FIG. 6(a-b). Red light-induced expression of GFP. (a) Representative overlaid brightfield and fluorescence images of GFP-expressing HEK293T cells. Cells were transduced with a lentiviral GFP expression vector and then co-transfected with the LID plasmids after 654-nm (0.2 mW/cm²) illumination or in the dark for 48 hours. (b) Comparison of GFP fluorescence intensities in fields-of-view (FOVs). Data represent mean values of 78 FOVs; error bars, standard error of the mean (SEM).

FIG. 7. (a) Two-state equilibrium model used in the thermodynamic simulation. (b) Thermographs showing the integration of heat transfer in simulated titration experiments.

FIG. 8. SDS-PAGE analysis of purified DrBphP and nanobodies. Proteins were purified by nickel affinity and SEC chromatography. To examine in vitro biotinylation efficiency by BirA, the biotinylated protein was bound to streptavidin beads (Dynabeads™ M-280 Streptavidin, Thermo Fisher Scientific) and the bound protein (lane 4) was compared with the input protein (lane 3). The lower band in the lane 4 was streptavidin released from the beads when boiling the sample in an SDS loading buffer.

FIG. 9(a-b). Structures and spectra of the dark and light forms of DrBphP. (a) Structures of DrBphP dark and light forms previously reported showing the biliverdin chromophore bound to a tri-domain photosensory module and conformational changes of a tongue motif interacting with the biliverdin binding pocket. (b) Absorption spectra of the dark and light states of the biotinylated DrBphP after the 775-nm (0.3 mW/cm², 10 min) and 654-nm (0.5 mW/cm², 2 min) illuminations, respectively.

FIG. 10(a-b). Column chromatography-based phage display selection. (a) Two-step biopanning FPLC setup. (b) Flow rate and illumination time setup. In the Step 1, 2 mL phage-displayed nanobodies were loaded to two connected transparent glass columns (HR 5/5, GE Healthcare) packed with 0.4 and 0.2 mL streptavidin agarose resin (Pierce). Before divided into the two columns, the resin was incubated with 1.2 mL 20 μM biotinylated DrBphP in the dark for 30 min. Next, DrBphP in the first (negative selection) and second (positive selection) columns were converted to the dark and light forms by the 775-nm (0.3 mW/cm², 10 min) and 654-nm (0.5 mW/cm², 2 min) illumination, respectively. After the phage injection, the flow rate was set to be 0.04 mL/min and then decreased to 0 when the UV 280 nm baseline was stable (i.e., non-bound phages were washed out). In the Step 2, the first column was removed, and phages were eluted from the second column by the 775-nm (0.8 mW/cm²) illumination for a given time. A pre-elution fraction was collected as a control for the phage count comparison with a light elution fraction to estimate the ratio of phages specifically eluted by the light to those non-specifically eluted (refer to Table 3).

FIG. 11. Yeast two-hybrid screening. Phage display-enriched nanobodies, as preys, were subcloned to pGADT7 encoding a GAL4 AD domain. DrBphP, as a bait, was inserted to pGBKT7 encoding a GAL4 DNA-binding domain. The right panel shows a representative result of two replica spotted plates incubated in the dark or under the 654-nm illumination.

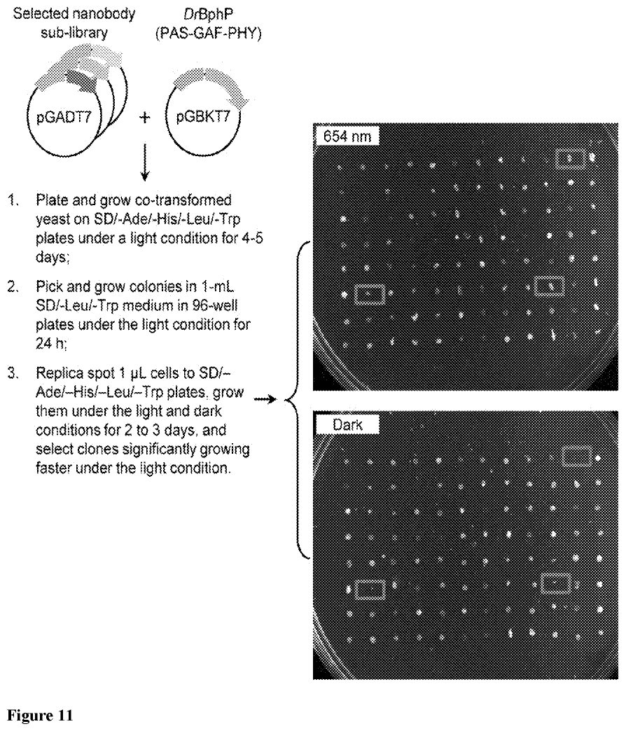

FIG. 12. In-gel detection of fluorescently labelled nanobodies expressed in HEK293T cells. Cells were transiently transfected with plasmids encoding SNAP-tagged nanobody fusions. Proteins in supernatants of sonication-lysed cells were specifically labeled with SNAP-Surface 649 and analyzed by SDS-PAGE and fluorescence imaging with an Odyssey CLx™ imaging system.

FIG. 13(a-b). Analytical SEC analyses of (a) nanobodies at different concentrations and (b) DrBphP in the light and dark forms. Proteins were loaded to a Superdex™ 200 Increase 10/300 GL column pre-equilibrated with 1×PBS and eluted at a flow rate of 0.75 mL/min at 4° C.

FIG. 14(a-c). ITC analysis of the titration of (a) 80 μM LDB-3 or (b) 50 μM LDB-14 into 1×PBS buffer, and (c) the titration of 1×PBS buffer into 10 μM DrBphP. The raw data (top) and the integration of heats (bottom) for each titration are shown.

FIG. 15(a-b). BLI analysis of LDB-3 and LDB-14 binding kinetics. BLI sensorgrams show DrBphP binding to LDB-3 (a) and LDB-14 (b). Nanobodies were immobilized on Streptavidin biosensors and interacted with DrBphP after the 654-nm (light form) or 775-nm (dark form) illumination. Data were fitted using a global 1:1 model.

FIG. 16(a-b). Inhibition of DrBphP relaxation to the dark form by the nanobody binding. (a) Representative absorption spectra of the photoconverted light and dark forms and after the thermal relaxation by 775-nm illumination with or without LDB-3 or LDB-14 binding. (b) Time-course analysis of thermal relaxation rates of unbound and nanobody-bound light-form DrBphP by the 775-nm illumination. 400 μl 5 μM (final concentration) light-form DrBphP (after the 654-nm illumination at 0.5 mW/cm² for 2 min) was incubated with 5 μM (final concentration) LDB-3 or LDB-14 for 10 min before the thermal relaxation.

FIG. 17. Time-course analysis of red light-induced luciferase expression. HEK293T cells were co-transfected with the bait, prey, and GAL4UAS-luciferase reporter plasmids (~0.25 μg each) in a 0.5 mL culture. Transfected cells were incubated under the 654-nm (0.2 mW/cm²) illumination or in the dark. Data represent mean values of 3 measurements; error bars, standard deviation.

FIG. 18. Flowchart and timeline of COMBINES-LID.

### DETAILED DESCRIPTION

In a first aspect, the disclosure provides Deinococcus radiodurans bacteriophytochrome (DrBphP) light form-binding antibodies, comprising a set of complementarity-determining regions (CDRs) selected from the group consisting of a CDR1, CDR2, and CDR3 combination selected from the group consisting of:
(a) SEQ ID NO:1 (CDR1), SEQ ID NO:2 (CDR2), and SEQ ID NO:3 (CDR3); or
(b) SEQ ID NO:4 (CDR1), SEQ ID NO:5 (CDR2), and SEQ ID NO:6 (CDR3).

Table 6 provides the CDR sequences.

TABLE 6
CDR sequences of light-induced
dimerization (LID) binder clones

| Row | IDs | CDR1 | CDR2 | CDR3 |
|---|---|---|---|---|
| 1 | LDB-3 | FTWDHYI | ENGDAWN | IGFDVPSGR |
| | | (SEQ | (SEQ ID | SWQGSHFWM |
| | | ID | NO: 2) | (SEQ ID |
| | | NO: 1) | | NO: 3) |
| 2 | LDB-14 | TTSRWES | WQNNSVP | AQHNFLGHR |
| | | (SEQ ID | (SEQ ID | (SEQ ID |
| | | NO: 4) | NO: 5) | NO: 6) |

As described herein, the antibodies herein can be used, for example, as the effector protein in a light-induced dimerization (LID) system as described in detail in the examples that follow. As detailed in therein, a light inducted dimerization (LID) system comprises two separate proteins or domains which serve as a sensor and an effector (dimerization binder). The antibodies of the present disclosure can, for example, serve as the effector protein when a sensor comprises the photosensory module of Deinococcus radiodurans bacteriophytochrome (DrBphP). The antibodies of the present disclosure specifically bind to DrBphP in its light form, and can be used, in non-limiting examples, to control light activated gene expression and spatiotemporal activation of chimeric antigen receptor T (CAR-T) cells.

As disclosed herein, “antibody” refers to immunoglobulin molecules and immunologically active portions of immunoglobulin molecules, i.e., molecules that contain the one of the listed CDR sets and binds to DrBphP in its light form, as described in detail in the examples. As such, the term antibody encompasses not only whole antibody molecules, but also antibody fragments as well as variants (including derivatives) of antibodies and antibody fragments. Such antibody or antibody fragments thereof may include, but are not limited to a monoclonal antibody, humanized antibody, chimeric antibody, Fab′, F(ab′)₂, Fab, Fv, rIgG, recombinant single chain Fv fragments (scFv), single-domain antibody (nanobody), bivalent or bispecific molecule, diabody, triabody, and tetrabody, or fragments thereof. In one specific embodiment, the antibody comprises a single-domain antibody. In any embodiment herein, the antibodies may be recombinant antibodies.

The antibodies may comprise a suitable scaffold sequence by which to appropriately present the 3 CDRs. Any scaffold sequence may be used, as deemed suitable for an intended use. In one embodiment, the scaffold comprises an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the following amino acid sequence:

(SEQ ID NO: 46)
EVQLQASGGGFVQPGGSLRLSCAASG - (CDR1) -
(SEQ ID NO: 47)
MGWFRQAPGKEREFVSAIS - (CDR2) -
(SEQ ID NO: 48)
YYADSVKGRFTISRDNSKNTVYLQMNSLRAEDTATYYCA - (CDR3) -
(SEQ ID NO: 49)
YWGQGTQVTVSS.

In another embodiment, the scaffold comprises an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the following amino acid sequence:

(SEQ ID NO: 50)
EVX1LQASGGGFX2X3PGGSLRLSX4AASG - (CDR1) -
(SEQ ID NO: 51)
MGWX5RQX6PX7KEREFVSAIS - (CDR2) -
(SEQ ID NO: 52)
YYX8DX9VKGRFTISRDNX10KNTX11YLQMX12SLX13X14EDTA
X15YYCX16 - (CDR3) -
(SEQ ID NO: 49)
YWGQGTQVTVSS;

wherein
X1 is K, Q, N, R, D, or E;
X2 is D, G, E, A, V, L, or I;
X3 is L, F, W, G, A, V, L, or I;
X4 is K, R, D, E, N, or Q,
X5 is V, P, G, A, L, I, or M;
X6 is T, A, S, G, V, L, or I;
X7 is E, G, D, A, V, L, or I;
X8 is P, A, M, G, V, L, or I;
X9 is T, or S;
X10 is A, S, M, G, V, L, or I;
X11 is L, V, G, A, or I;
X12 is S, N, Q, or T;
X13 is K, R, D, or E;
X14 is S, A, T, G, V, L, or I;
X15 is M, T, V, L, or I;
X16 is V, G, L, I, or A.

In each of these embodiments, the CDR1, CDR2, and CDR3 is a set of CDRs provided in Table 6 (i.e.: CDR1 is SEQ ID NO:1, CDR2 is SEQ ID NO:2, and CDR3 is SEQ ID NO:3; or CDR1 is SEQ ID NO:4, CDR2 is SEQ ID NO:5, and CDR3 is SEQ ID NO:6). The defined sequences comprise a camelid-based universal scaffold, with the three variable complementarity-determining regions (CDRs) interspersed.

In one embodiment the percent identity requirement is based only on the scaffold sequence (i.e.: not including identity with the CDR sequence). In another embodiment, the percent identity requirement is based on the scaffold sequence and a set of CDRs.

In one embodiment, the antibody comprises an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical along the length of the amino acid sequence of SEQ ID NO:9 or SEQ ID NO:10.

| | | |
|---|---|---|
| LDB-3 | EVQLQASGGGFVQPGGSLRLSCAASGFTWDHYIMGWFRQAPGKEREFVSAISENGDAWNY | 60 |
| LDB-14 | EVQLQASGGGFVQPGGSLRLSCAASGTTSRWESMGWFRQAPGKEREFVSAISWQNNSVPY | 60 |
| | ************************** ******************* * | |
| | CDR3 | |
| LDB-3 | YADSVKGRFTISRDNSKNTVYLQMNSLRAEDTATYYCAIGFDVPSGRSWQGSHFWMYWGQ | 120 |
| LDB-14 | YADSVKGRFTISRDNSKNTVYLQMNSLRAEDTATYYCAAQH------NFLGH---RYWGQ | |
| | *************************************** *** | |
| LDB-3 | GTQVTVSS 128 (SEQ ID NO: 9) | |
| LDB-14 | GTQVTVSS 119 (SEQ ID NO: 10) | |

The antibodies can tolerate significant substitutions in the scaffold residues. In some embodiments, a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In all of these embodiments, the percent identity requirement does not include any additional functional domain that may be incorporated in the polypeptide. In all embodiments, any N-terminal methionine residue is optional and may be present or absent, and is not considered when determining percent identity.

In another embodiment of any of the above embodiments, the antibodies further comprise one or more functional domains. As used herein, a “functional domain” is any polypeptide of interest that might be fused or covalently bound to the polypeptides of the disclosure. In non-limiting embodiments, such functional domains may comprise one or more polypeptide antigens, polypeptide therapeutics, enzymes (e.g., Cre recombinase), detectable domains (ex: fluorescent proteins or fragments thereof), DNA binding proteins, transcription factors, membrane receptors (e.g., T cell receptors) and their binding regulators, etc. The one or more functional domains may be fused at any appropriate regions within the polypeptides of the disclosure, including but not limited to at the N-terminus or at the C-terminus of the polypeptide.

In a further embodiment, a plurality of the antibodies are bound to a solid support. Any suitable solid support may be used, including but not limited to paper, nitrocellulose, beads, cell culture plates, nanoparticles, etc.

The antibodies of the disclosure may include additional residues at the N-terminus, C-terminus, internal to the polypeptide, or a combination thereof; these additional residues are not included in determining the percent identity of the polypeptides of the invention relative to the reference polypeptide. Such residues may be any residues suitable for an intended use, including but not limited to detectable proteins or fragments thereof (also referred to as “tags”). As used herein, “tags” include general detectable moieties (i.e.: fluorescent proteins, antibody epitope tags, etc.), therapeutic agents, purification tags (His tags, etc.), linkers, ligands suitable for purposes of purification, ligands to drive localization of the polypeptide, peptide domains that add functionality to the polypeptide of the disclosure.

The antibodies can include genetically coded and non-genetically coded amino acids, chemically or biochemically modified or derivatized amino acids, and modified peptide backbones. The recombinant antibodies may comprise L-amino acids+glycine, D-amino acids+glycine (which are resistant to L-amino acid-specific proteases in vivo), or a combination of D- and L-amino acids+glycine. The recombinant antibodies described herein may be chemically synthesized or recombinantly expressed. The recombinant antibodies may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

In another aspect, the disclosure provides nucleic acids encoding the antibody of any embodiment or combination of embodiments of the disclosure. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In another aspect, the disclosure provides expression vectors comprising the nucleic acid of any embodiment or combination of embodiments of the disclosure, operatively linked to a control sequence. “Expression vector” includes vectors that operatively link a nucleic acid coding region or gene to any control sequences capable of effecting expression of the gene product. “Control sequences” operatively linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In a further aspect, the disclosure provides cells that comprises the antibody, nucleic acid, or expression vector (i.e.: episomal or chromosomally integrated) of any embodiment or combination of embodiments. The host cells can be either prokaryotic or eukaryotic.

In another aspect, the disclosure provides kit, comprising:
(a) the antibody, nucleic acid, expression vector, and/or cell of any of any embodiment or combination of embodiments disclosed herein; and
(b) a photosensory module of Deinococcus radiodurans bacteriophytochrome (DrBphP), including but not limited to a polypeptide at least 70%, 75%, 80%, 85%, 90%, 91%, 92%, 93%, 94%, 95%, 96%, 97%, 98%, 99%, or 100% identical to the amino acid sequence of SEQ ID NO: 11, wherein optional residues in parentheses may be present or absent and are not considered when determining the percent identity when absent, a nucleic acid encoding such a photosensory module of DrBphP, an expression vector comprising the nucleic acid encoding such a photosensory module of DrBphP operatively linked to a control sequence, and/or cell comprising the photosensory module of DrBphP, the nucleic acid encoding the photosensory module of DrBphP, and/or the expression vector comprising the nucleic acid encoding a photosensory module of DrBphP operatively linked to a control sequence.

Any suitable expression vector may be used as deemed appropriate for an intended purpose, including but not limited to pcDNA3 or pBAD plasmids. Any suitable cell may be used as deemed appropriate for an intended purpose, including but not limited to HEK293T cells.

### Exemplary DrBphP Sensory Module

(SEQ ID NO: 11)
(M)SRDPLPFFPPLYLGGPEITTENCEREPIHIPG
SIQPHGALLTADGHSGEVLQMSLNAATFLGQEPTV
LRGQTLAALLPEQWPALQAALPPGCPDALQYRATL
DWPAAGHLSLTVHRVGELLILEFEPTEAWDSTGPH
ALRNAMFALESAPNLRALAEVATQTVRELTGFDRV
MLYKFAPDATGEVIAEARREGLHAFLGHRFPASDI
PAQARALYTRHLLRLTADTRAAAVPLDPVENPQTN
APTPLGGAVLRATSPMHMQYLRNMGVGSSLSVSVV
VGGQLWGLIACHHQTPYVLPPDLRTTLEYLGRLLS
LQVQVKEAADVAAFRQSLREHHARVALAAAHSLSP
HDTLSDPALDLLGLMRAGGLILRFEGRWQTLGEVP
PAPAVDALLAWLETQPGALVQTDALGQLWPAGADL
APSAAGLLAISVGEGWSECLVWLRPELRLEVAWGG
ATPDQAKDDLGPRHSFDTYLEEKRGYAEPWHPGEI
EEAQDLRDTLTGAL

As described herein, the kits disclosed can be used, for example, as a light-induced dimerization (LID) system. As detailed in the examples, an LID system comprises two separate proteins or domains which serve as a sensor and an effector (dimerization binder). The antibodies of the present disclosure can, for example, serve as the effector protein when a sensor comprising the photosensory module of Deinococcus radiodurans bacteriophytochrome (DrBphP). The polypeptides of the present disclosure specifically bind to DrBphP in its light form, and can be used, in non-limiting examples, to optogenetically control CAR-T cell activation in specific organ and tissue regions, such as a solid tumor region, the LID system can be used to control the expression of chimeric TCR genes or the interaction of the chimeric TCR and a costimulatory domain, such as CD3ζ, to activate the TCR signaling pathway by separately fusing the photoreceptor and the antibody to the TCR and the costimulatory domain.

In one aspect, the disclosure provides methods for use of the antibodies, kits, nucleic acids, expression vectors, or host cell of any embodiment or combination of embodiments disclosed herein for any suitable purpose, including but not limited to use of an LID system for any purpose, including but not limited to control light activated gene expression and spatiotemporal activation of chimeric antigen receptor T (CAR-T) cells, or precisely controlling the split Cre recombinase in genetically modified animals, etc.

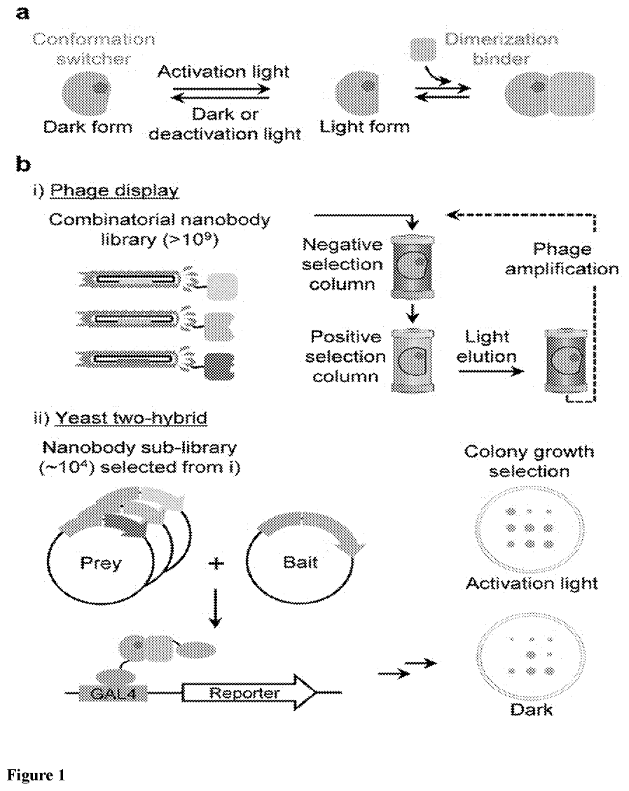

In another aspect, the disclosure provides methods for making light induced dimerization (LID) systems. The methods are described in detail in the examples, and comprise:
(1) phage display to enrich binders (such as nanobodies) that only bind to the light form of a conformation switcher (i.e.: changes conformation when exposed to light); and
(2) yeast two-hybrid (Y2H) screening of the enriched sub-library to select for in vivo activity (See FIG. 1b).

Such LID systems may be constructed using polypeptides selected from a vastly diverse synthetic combinatorial library. The combinatorial library may comprise DNA sequences chemically synthesized by a combinatorial method, such as a trinucleotide mutagenesis technology (PMID: 7838712), to obtain the diversity higher than 10′. The DNA sequences may encode polypeptides consisting of a consensus sequence, typically providing a structural scaffold, such as an immunoglobulin, non-immunoglobulin (PMID: 25931178), or computationally designed scaffold, and variable loop sequences, similar to complementarity determining regions (CDRs) of antibodies, which are structurally flexible and thus can be rationally randomized by incorporating a combination of amino acids under a given ratio into each loop sequence positions. Since both the polypeptides that form a LID system can be selected from a combinatorial library, this selection method may be applicable to generate LID systems for a wide range of light-induced conformation switching sensor molecules. These methods are a significant improvement over prior methods which rely on the using naturally occurring LID systems and their derivatives with intrinsic limitations, such as complex structures and the high dark activity, which requires further engineering of protein structures and activities.

In one embodiment, the methods comprise
(a) screening a synthetic combinatorial polypeptide library with a light form of a conformation switcher to identify binding polypeptides; and
(b) screening a synthetic combinatorial polypeptide library with a dark form of a conformation switcher (conformation assumed when not exposed to light) to identify dimerization polypeptides in the library that (i) bind to the light form of a conformation switcher, and (ii) do not bind to the dark form of a conformation switcher. In one embodiment, the polypeptide library may comprise a synthetic combinatorial immunoglobulin, non-immunoglobulin, or computationally designed library, such as a single-domain antibody library. In another embodiment, the screening in step (a) is carried out 1, 2, 3, 4, 5, 6, or more times. In a further embodiment, the screening in step (b) is carried out 1, 2, 3, 4, 5, 6, or more times. The methods may further comprise any other suitable steps, including but not limited to any step or combination of steps disclosed in the examples.

### EXAMPLES

Summary: Protein dimerization systems that can be controlled by red light with increased tissue penetration depth are a highly needed tool for clinical applications such as cell and gene therapies. However, existing red light-induced dimerization systems are all based on phytochrome photoreceptors and naturally occurring binding partners with complex structures and suboptimal in vivo performance, limiting mammalian applications. Here, we introduce an efficient, generalizable method combinatorial binders-enabled selection of light induced dimerization (COMBINES-LID) for creating highly specific light-induced dimerization systems. It involves a two-step binder screen (phage display and yeast two-hybrid) of a combinatorial nanobody library to obtain binders that selectively engage a light-activated form of a photoswitchable protein or domain not the dark form. Proof-of-principle was provided by creating nanobody-based, red light-induced dimerization (nanoReD) systems comprising a truncated bacterial phytochrome sensory module using a mammalian endogenous chromophore, biliverdin, and a light-form specific nanobody. Selected nanoReD systems were biochemically characterized and exhibited low dark activity and high induction specificity for in vivo activation of gene expression. Overall, COMBINES-LID opens new opportunities for creating genetically encoded actuators for the optimal manipulation of biological processes

Different from single-component actuator systems such as microbial opsins, light-induced protein dimerization (LID) systems comprises two separate proteins or domains which serve as a sensor and an effector. The sensory function is initiated by i) light-induced chromophore isomerization or chromophore-protein bond formation triggering a conformational change of a chromophore-bound photosensory protein (hereafter named ‘conformation switcher’), or ii) photolytic release of a caged ligand or isomerization of a photoswitchable ligand that serves as a dimerization inducer. Naturally occurring conformation switchers widely exist in all kingdoms of life and many have been identified and characterized in the past three decades (Table 1). They have diverse structural and optical properties, offering flexible choices for in vivo applications. Many use widely shared metabolites from bacteria to humans as chromophores; for example, riboflavin-5′-phosphate bound to light-oxygen-voltage (LOV) sensing domains and biliverdin, a heme-derived linear tetrapyrrole found in bacteriophytochrome (BphP).

TABLE 1
Summary of photoswitchable proteins or domains that can potentially
be used as conformational switchers in LID systems.

| Photo- | | | | | | | Natural | |
|---|---|---|---|---|---|---|---|---|
| switchable | | | | | Oligomeric state | | light | |
| proteins (or | | | Excitation | Reversion | Dark | Light | induced | |
| domains) | Example(s) | Chromophore(s) | λ (nm) | λ (nm) | form | form | binder(s) | Reference(s) |
| UV receptors | UVR8 | Trp | ~300 | Dark | Dimer | Monomer | COP1 | 5-7 |
| Cyanobacterio- | CcaS | PCB | ~535 | ~672/dark | Monomer | Monomer | CcaR | 8-9 |
| chromes | cPAC | PCB | ~410 | ~520/dark | Dimer | Dimer | Unknown | 10 |
| | UirS | PCB | ~400 | ~530/dark | Monomer | Monomer | UirR | 11-12 |
| Sensors of | PixD | FAD or FMN | ~450 | Dark | Decamer | Dimer | PixE | 13-15 |
| blue-light | bPAC | FAD or FMN | ~450 | Dark | Dimer | Dimer | Unknown | 16-18 |
| using FAD | | | | | | | | |
| (BLUF) | | | | | | | | |
| domains | | | | | | | | |
| LOV domains | AsLOV2 | FMN | ~450 | Dark | Monomer | Monomer | Unknown | 19-21 |
| | YtvA | FAD, FMN, or | ~450 | Dark | Dimer | Dimer | Unknown | 22-24 |
| | | riboflavin | | | | | | |
| | VVD | FAD or FMN | ~450 | Dark | Monomer | Dimer | VVD | 25-27 |
| | FKF1 | FMN | ~450 | Dark | Monomer | Dimer | GI | 28-30 |
| | EL222 | FMN | ~450 | Dark | Monomer | Dimer | Unknown | 31-32 |
| Cryptochromes | CRY2 | FAD | ~450 | Dark | Monomer | Monomer | CIB1 | 33-34 |
| Fluorescent | Dronpa1 | p-HBI | ~400 | ~500/dark | Monomer | Dimer | Unknown | 35-36 |
| protein | 45K/N | | | | | | | |
| domains | PYP | p-coumaric | ~450 | Dark | Monomer | Monomer | Unknown | 37-38 |
| | | acid | | | | | | |
| Opsins | BeCycl | Retinal | ~530 | Dark | Dimer | Dimer | Unknown | 39 |
| | Op | | | | | | | |
| Cobalamin | TtCBD | AdoCbl, | ~545 | Dark | Tetramer | Monomer | Unknown | 40 |
| binding | | MetCbl, or | | | | | | |
| domains | | CNCbl | | | | | | |
| (CBDs) | MxCBD | AdoCbl, | ~545 | Dark | Tetramer | Monomer | Unknown | 40 |
| | | MetCbl, or | | | | | | |
| | | CNCbl | | | | | | |
| Phytochromes | RpBphP1 | BV | ~740 | ~636/dark | Dimer | Dimer | PpsR2 | 41-42 |
| | DrBphP | BV | ~655 | ~780/dark | Dimer | Dimer | Unknown | 4, 43-44 |
| | Cph1 | PCB or | ~657 | ~731/dark | Dimer | Dimer | Unknown | 45-47 |
| | | PΦB | | | | | | |
| | PhyB | PCB | ~660 | ~740/dark | Monomer | Monomer | PIF3/PIF6 | 48-50 |

The effector function of LID is executed by a ‘dimerization binder’ which specifically binds to the conformation switcher in its light form—the state after a light-induced conformational change occurring to its thermally stable state in the dark, or the dark form (FIG. 1a). Many natural conformation switchers do not modulate protein-protein interaction and are not associated with any known dimerization binders; for example, in phytochromes, conformation switcher domains (known as a photosensory module) are typically fused with an enzymatic domain or module to allosterically regulate catalytic activity. Although a few natural dimerization binders have been identified (Table 1), they are limited by the basal activity in the dark (or dark activity) and other undesirable properties. It is yet difficult to design new dimerization binders with suitable specificity, sensitivity, and kinetics.

For deep-tissue applications in animals, LID is required to sense an optical input in the 650-900 nm region, known as a tissue transparency window, because tissue absorbance, autofluorescence, and light scattering are minimized in this region.

### Results and Discussion

Screening of Dimerization Binders for DrBphP. We devised a two-step screening method, combinatorial binders-enabled selection of LID (COMBINES-LID), which involves phage display to enrich binders that only bind to the BphP light form and then yeast two-hybrid (Y2H) screening of the enriched sub-library to select for in vivo activity (FIG. 1b). A high-quality synthetic combinatorial nanobody library generated in our previous work was used. These nanobodies have an optimized scaffold and rationally randomized CDRs with an estimated sequence diversity of 1.23 to 7.14×10⁹.

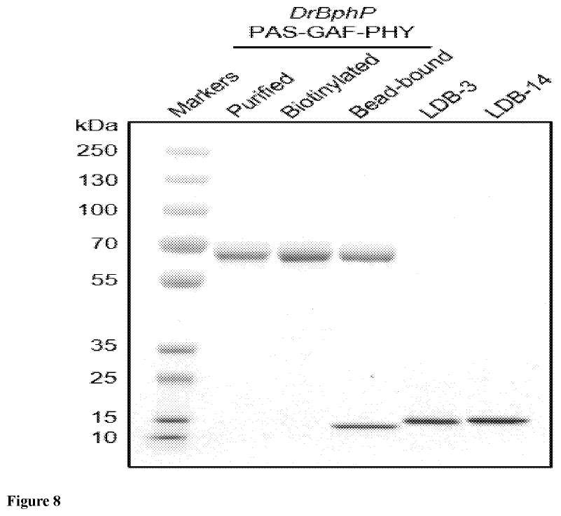

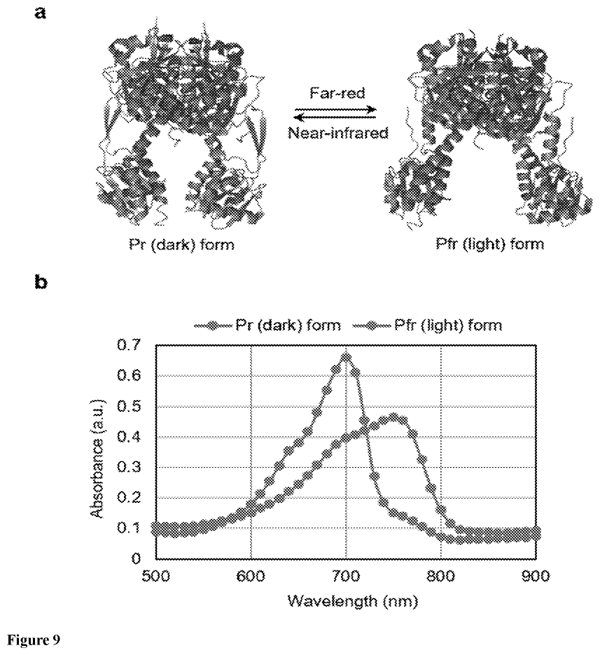

To simplify the structure of the conformation switcher, the photosensory module of Deinococcus radiodurans bacteriophytochrome (DrBphP) was chosen. Its light and dark forms can be photoconverted by activating far-red (e.g., 654 nm) and deactivating NIR (e.g., 775 nm) illuminations, respectively, and then stably maintained in a screening assay. The photoswitching efficiency is close to that of the full-length DrBphP. By contrast, the excised module of RpBphP1 showed impaired photoconversion.¹⁰ The tridomain module (hereafter named DrBphP for simplicity) comprising a Per-ARNT-Sim (PAS), a cGMP phosphodiesterase-adenylate cyclase-FhlA (GAF), and a phytochrome-specific (PHY) domains was expressed as a ~60 kDa fusion bearing a C-terminal AviTag and HisTag, incubated with biliverdin, purified, and biotinylated (FIG. 8) to serve as a bait for phage display. The photoconversion of the purified protein was confirmed by measuring the spectra of the light and dark forms (FIG. 9).

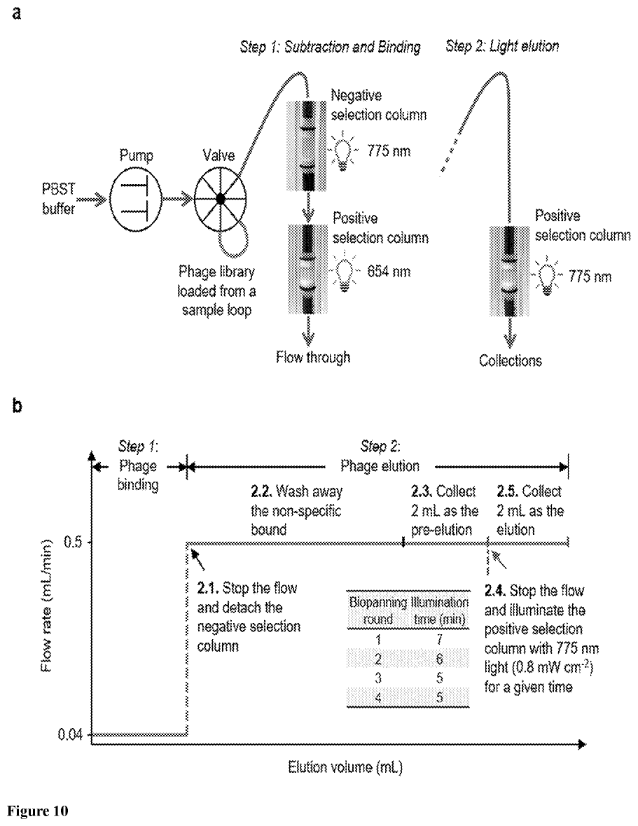

We hypothesized that specific and reversible dimerization binders are critical for the in vivo performance of LID, such as a low dark activity. To enhance selection efficiency, we used column chromatography to continuously separate phage-displayed nanobodies between the stationary and mobile phases as they passed through a column. Binding specificity was selected by loading the library onto two connected transparent columns, the first (negative selection) preloaded with biotinylated DrBphP in the dark form and the second (positive selection) with the light form (FIG. 10a). Thus, nanobodies captured in the second column should have zero or very low affinity to the dark form. Next, reversible binders were collected by eluting only dissociated nanobodies from the second column after switching the light to dark form by the 775-nm illumination. After four-round phage binding and elution with gradually decreased illumination times (FIG. 10b), the final eluent was estimated to contain ~90% light-eluted clones (Table 2) with the sequence diversity of ~10⁴.

TABLE 2
Enrichment of phage titers following each round of
biopanning for the dimerization binder selection.

| | | Pre-elution | 775 nm light |
|---|---|---|---|
| Round | Input count | count* | elution count** |
| 1 | ~1 × 10¹⁴ | ~5.4 × 10⁶ | ~2.0 × 10⁶ |
| 2 | ~1 × 10¹³ | ~1.2 × 10⁵ | ~2.3 × 10⁵ |
| 3 | ~1 × 10¹³ | ~1.8 × 10⁵ | ~7.3 × 10⁵ |
| 4 | ~1 × 10¹³ | ~1.5 × 10⁷ | ~1.4 × 10⁸ |

Note:
After phage binding, the positive selection column was washed with ~30 mL 0.05% PBST buffer. 2 mL pre-elution fraction (*) was collected at 0.5 mL/min immediately before the 775 nm-illumination at 0 mL/min. 2 mL elution fraction (**) was collected at 0.5 mL/min immediately after the illumination. Phage titers of the collected fractions were measured to determine the enrichment of clones specifically eluted by the light for each selection round.

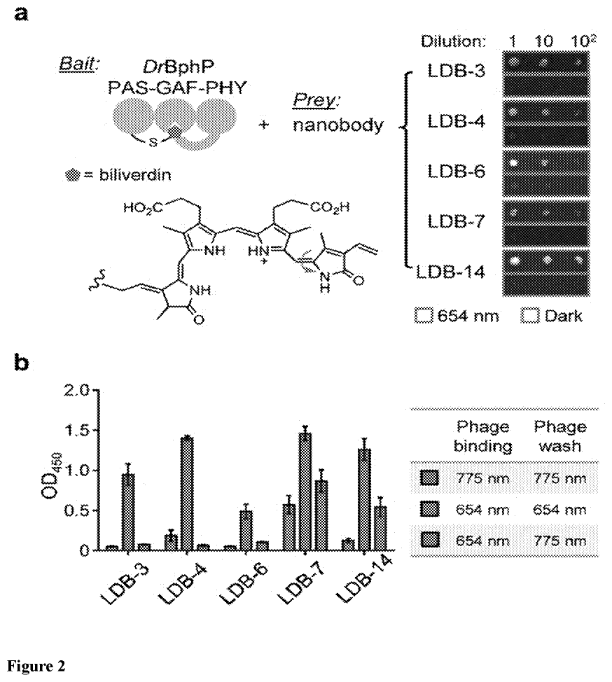

In vitro selected nanobodies were subcloned into a Y2H sub-library for the cell-based screening of cytoplasmic expression and binding specificity. Y2H was selected for the sub-library screening due to its suitable throughput and cost-effectiveness. Y2HGold cells were co-transformed with plasmids carrying genes of DrBphP and nanobodies and selected on SD/-Ade/-His/-Leu/-Trp agar plates under the 654-nm illumination. ~2,000 fully grown colonies were picked, inoculated into 1-mL SD/-Leu/-Trp medium, and replica spotted onto the agar plates to compare colony growth under the illumination and in the dark (FIG. 114). Five candidates grew only under the illumination (FIG. 2a) and with diverse CDR sequences (Table 3) were selected for further characterization.

TABLE 3
CDR sequences of light-induced dimerization
binders (LDBs) characterized in the work.

| | | | | SEQ ID |
|---|---|---|---|---|
| Nanobody | CDR1 | CDR2 | CDR3* | NOs: |
| LDB-3 | FTWDHYI | ENGDAWN | IGFDVPSGR | 1, 2, 3 |
| | | | SWQGSHFWM | |
| LDB-4 | DTSYLYS | WWWNLTQ | WSIYFPPGN | 12, 13 ,14 |
| | | | DYNGYH | |
| LDB-6 | FFSNWSD | FWADGTE | WYGPVNGFY | 15, 16, 17 |
| | | | MFD | |
| LDB-7 | STSDFES | SWFTNPP | HRSIWYHPT | 18, 19, 20 |
| LDB-14 | TTSRWES | WQNNSVP | AQHNFLGHR | 4, 5, 6 |

To confirm the binding specificity and reversibility, we assayed selected nanobodies by single phage enzyme-linked immunosorbent assay (ELISA). Phage displayed-nanobodies were first bound to the dark and light forms of biotinylated DrBphP immobilized in streptavidin-coated microtiter plates. To maintain the dark or light form, or to convert the light to dark form, the plates were under 654- and/or 775-nm illumination during the phage binding and wash steps. As expected, all candidates showed light-form binding specificity with non-detectable (LDB-3 and LDB-6) to relatively low (LDB-4, LDB-7, and LDB-14) binding to the dark form (FIG. 2b). Bound nanobodies were almost completely (LDB-3, LDB-4, and LDB-6) or partially (LDB-7 and LDB-14) washed off after converting the light to dark form.

Specificity Validation in a Mammalian Cell line. To determine whether nanobody candidates are suitable for mammalian applications, we tested their expression in human embryonic kidney 293T (HEK293T) cells. It is known that the same protein-protein interaction (PPI) found in yeast might not be detected in mammalian cells due to protein expression or stability issues; for example, a recent comparison of PPI assays in different hosts found that only half of human PPIs detected in yeast were also seen in HEK293T, and vice versa.²⁴ Thus, we were interested to know the success rate of nanobodies selected by COMBINES-LID that can be functionally expressed in mammalian cells.

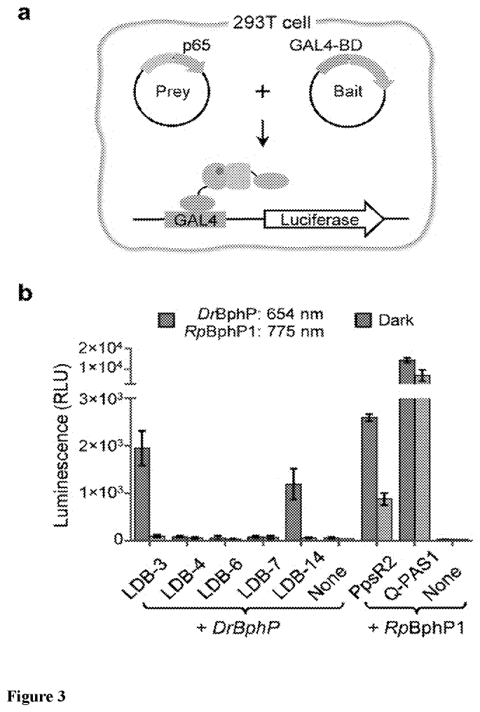

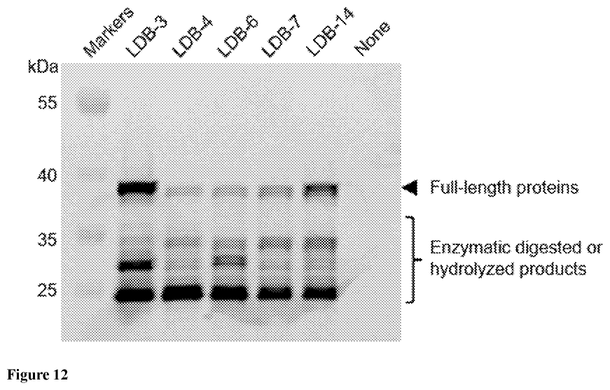

To compare in vivo activity, we assayed proteins by mammalian two-hybrid (M2H)²⁵ under a standardized condition. Specifically, DrBphP was fused with an N-terminal GAL4 DNA binding domain (BD) and nanobodies with a C-terminal p65 transcriptional activation domain (AD) to control the expression of a firefly luciferase reporter (FIG. 3a). After transient co-transfection with the BD and AD, and reporter plasmids, cells were cultured for 24 hours in the dark to express the DrBphP and nanobodies, and then maintained in the dark or under the 654-nm illumination for another 24 hours to compare the luciferase expression. Dark activity and specificity were analyzed by comparing the dark expression with a negative control (i.e., only DrBphP was expressed) and the light-induced expression with the dark expression, respectively. All candidates showed low dark activities, and the dark expression levels were close to the control (FIG. 3b). LDB-3 and LDB-14 showed the high specificity; their light-induced expression levels were increased by ~19.5 and ~19.1 folds, respectively. The other three candidates did not show obvious light activation. To understand their loss of the expected activity, we investigated protein stability in HEK293T. Specifically, all nanobodies bearing a C-terminal SNAP-tag were expressed, fluorescently labelled, and analyzed by sodium dodecyl sulfate gel electrophoresis (SDS-PAGE) to detect full-length proteins and degraded forms. All nanobodies were found with degraded fragments; however, compared with LDB-3 and LDB-14, the other three nanobodies showed drastically decreased levels of full-length proteins, suggesting that they might not be stable in the host cells (FIG. 12).

We compared the specificity of the nanobody-based LIDs with the RpBphP1-PpsR2 and RpBphP1-Q-PAS1 systems by M2H The RpBphP1-based LIDs showed light-enhanced expression (FIG. 3b). Light-induced expression was increased by ~2.95 and ~2.02 folds. As suggested by a mathematical model of LID, the specificity is determined by not only relative binding affinities of dimerization binders to dark and light forms of a conformation switcher, but also by their effective cellular concentrations. Our comparison with the same transfection and culture condition suggests that LDB-3 and LDB-14 offer significantly enhanced dimerization specificity.

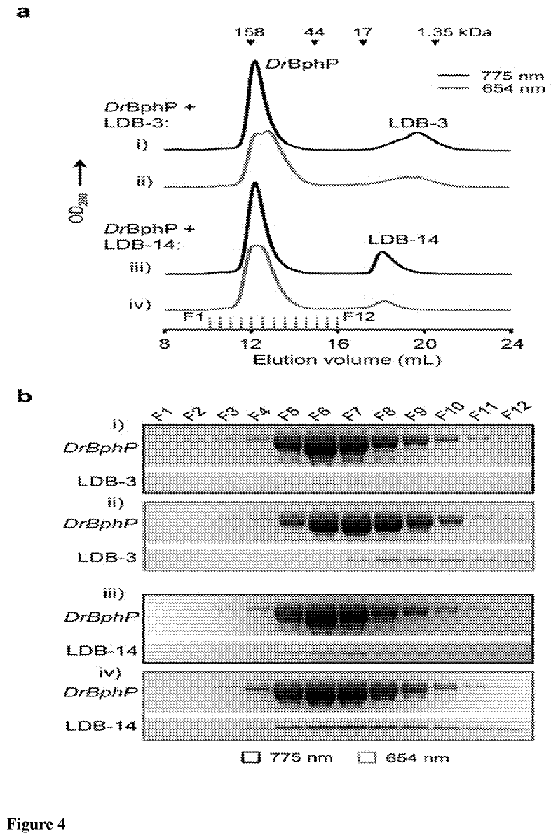

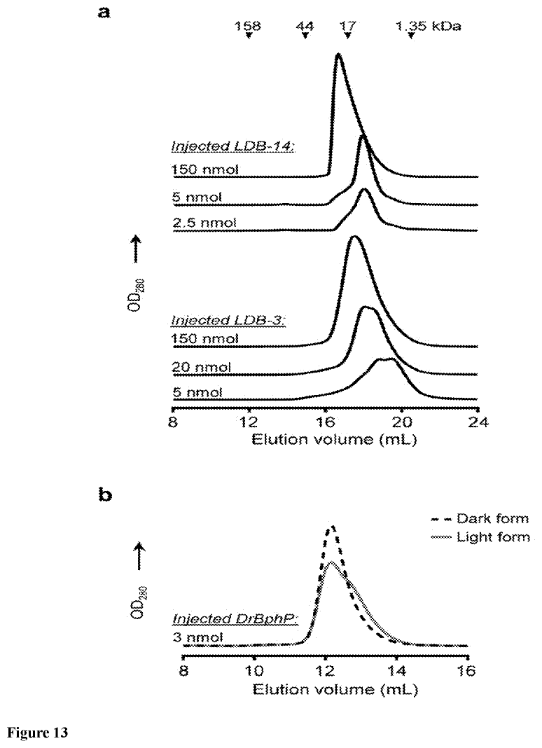

Biochemical Characterization of LDB-3 and LDB-14. We biochemically assessed selected nanobodies to understand their binding mechanisms. We first sought to detect light-induced DrBphP-nanobody complexes using analytical size-exclusion chromatography (SEC). LDB-3 and LDB-14 were bacterially expressed and purified with yields of ~2-3 milligrams per liter of culture. SEC data showed that LDB-3 was mixture of the monomer and dimer and LDB-14 mainly the monomer and both nanobodies dimerized at increased concentrations (FIG. 13a). Both the light and dark forms of DrBphP were eluted mainly as homodimers (FIG. 13b). After DrBphP was illuminated and then incubated with LDB-3 or LDB-14 in the dark, split SEC peaks of DrBphP were observed only for the light form (FIG. 4a), implying complex formation. Complexes were confirmed by SDS-PAGE detection of coeluted DrBphP and nanobodies (FIG. 4b). Interestingly, gel analysis revealed that LDB-3 was co-eluted with DrBphP later than LDB-14, suggesting that they might have different binding configurations or affinities. Consistent with the single phage ELISA result (FIG. 2b), LDB-14 appeared to weakly interact with the dark form since a faint nanobody band was detected in the dark-form DrBphP factions.

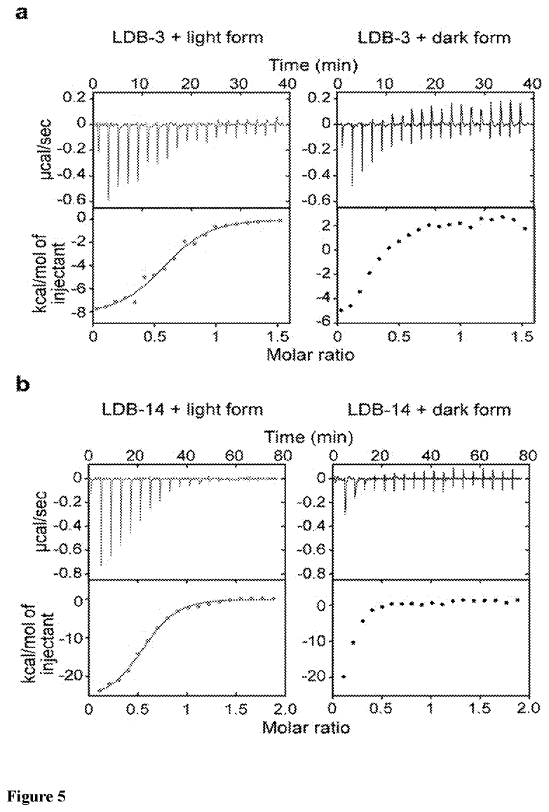

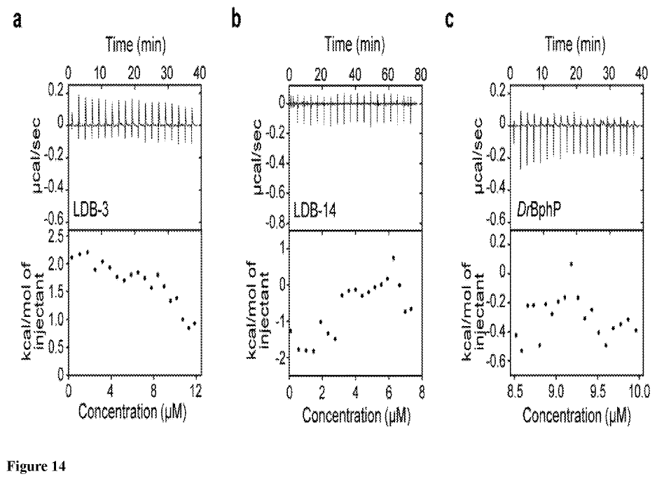

We next studied the thermodynamics of nanobody binding by isothermal titration calorimetry (ITC). Binding data obtained by titrating LDB-3 or LDB-14 into a photoconverted light-form sample were fitted using a one-site model (R²=~0.99) to give apparent dissociation constants (K_{D}^{app}s) of 1.01 and 0.47 μM, respectively (FIG. 5 and Table 4). The binding site number of DrBphP was calculated to be ~0.6, consistent with a previous small-angle X-ray scattering result that only 64% of the protein molecules in the photoconverted sample adopted the light-state conformation. Unexpectedly, the thermograph of LDB-3 titration into the dark form showed significant heat exchange: a clear transition from heat release to absorption when more LDB-3 was added, which was not observed for LDB-3 titration into the light form (FIG. 5a). This transition suggests that the exothermic binding of LDB-3 to the dark form might be coupled to an endothermic process, which is likely to be the LDB-3 dimer dissociation (FIG. 14). In other words, LDB-3 binding to the dark form might be inhibited by LDB-3 dimerization, thus providing a mechanism to explain the observed low dark activity of LDB-3 (refer to the thermodynamic simulation in Materials and Methods). LDB-14 was found to only bind to a small fraction (~15%) of DrBphP in the dark-form sample but did not generate measurable heat of binding with the major population (FIG. 5b). The minor fraction was likely the protein switched to the light form during our assay.

TABLE 4
ITC-derived thermodynamic parameters for LDB-3 and
LDB-14 binding to the DrBphP light form.

| | n | K_{D}^{app} | ΔH | ΔG | -TΔS |
|---|---|---|---|---|---|
| | (stoichiometry) | (μM) | (kJ/mol) | (kJ/mol) | (kJ/mol) |
| LDB-3 | 0.605 | 1.01 | −37.0 | −34.2 | 2.78 |
| LDB-14 | 0.556 | 0.47 | −112.8 | −36.1 | 76.7 |

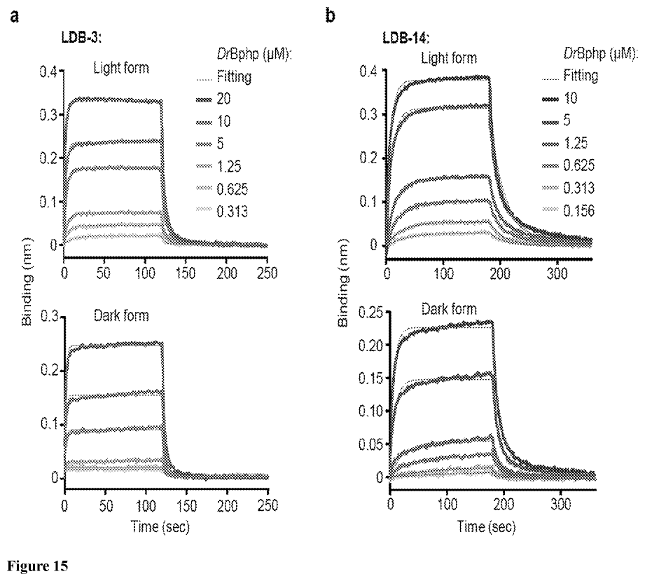

The binding kinetics of LDB-3 and LDB-14 were measured by Bio-Layer Interferometry (BLI). The assay was performed by incubating the light or dark form with nanobodies immobilized on streptavidin biosensors (refer to Supplementary Methods). The result revealed that, compared with LDB-14, LDB-3 has a weaker binding affinity to the light form mainly due to a ~4.9-fold faster dissociation from the DrBphP (k_{off}=−18.5×10⁻² s⁻¹) (FIG. 15 and Table 5). Theoretically, the fast dissociation could offer higher temporal resolution for the reversible control of LID. The BLI analysis of the dark form binding was not straightforward because the white light signal used by this optical technique might partially convert the biosensor-bound DrBphP to the light form.

TABLE 5
Kinetic parameters of selected dimerization binders
binding to the light and dark forms.

| | Molar | K_{D}^{app} | Kₒₙ^{app} | K_{off}^{app} |
|---|---|---|---|---|
| | ratio | (10⁻⁶ M) | (10⁴ M⁻¹ s⁻¹) | (10⁻² s⁻¹) |
| | LDB-3 | | | |
| Light form | 1:1 | 7.7 | 2.4 | 18.5 |
| Dark form* | 1:1 | 25 | 1.06 | 26.5 |
| | LDB-14 | | | |
| Light form | 1:1 | 2.4 | 1.56 | 3.74 |
| Dark form* | 1:1 | 10 | 0.607 | 6.07 |

Note:
*The dark form binding data are not reliable because the white light conducted to BLI biosensors might partially convert DrBphP to the light form.

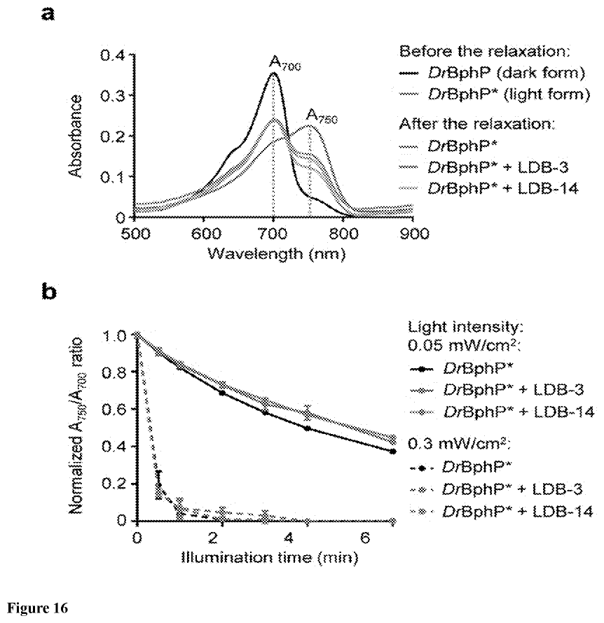

Since DrBphP photoconversion and nanobody binding might reciprocally affect each other, we asked whether the nanobody binding can slow the photoconversion to the dark state. To test this, we illuminated DrBphP with different exposure times and light intensities and measured the percentage of the dark state in the protein by the ratio of absorption at 750 nm (A₇₅₀) to 700 nm (A₇₀₀) (FIG. 16a). Compared with unbound DrBphP, the nanobody-bound DrBphP had decreased photoconversion rates (FIG. 16b), suggesting that the nanobody binding can stabilize the light state. At a relatively low light intensity (0.05 mW/cm²), the LDB-3-bound DrBphP had a slightly faster relaxation to the dark state than the LDB-14-bound DrBphP, likely due to the faster dissociation of the DrBphP-LDB-3 complex. Together, our biochemical data support the high specificity and reversibility of the nanobody-based LID systems.

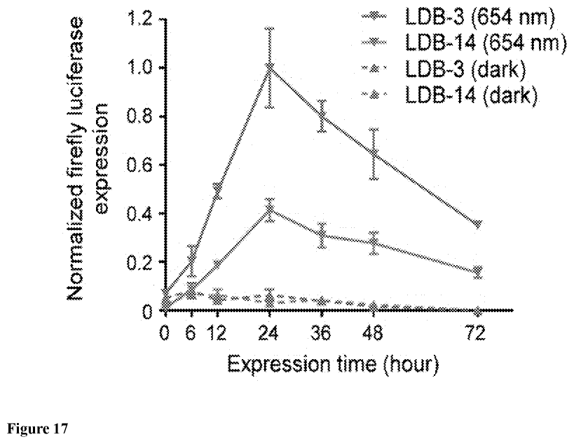

Red Light-Activated Gene Expression. To develop in vivo applications, we focused on light-induced gene expression. We first determined the time-course response of light-induced activation of luciferase expression in HEK293T cells. Under the same culture and transfection condition, luciferase levels with or without 654-nm illumination were measured at seven time points up to 72 hours. For both LDB-3 and LDB-14, luciferase levels after illumination reached half maximum and maximum at 12 and 24 hours, respectively (FIG. 17). The half-maximum and maximum luciferase levels in cells expressing LDB-3 were ~2.5-fold higher than those of cells expressing LDB-14, which seems to be correlated with in vivo nanobody stability (FIG. 12).

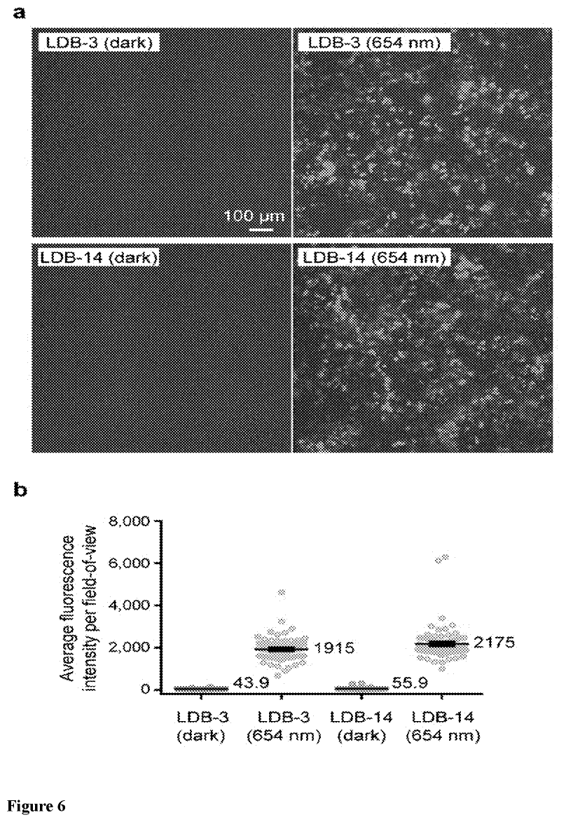

The luciferase assay required releasing the protein by cell lysis, so we also measured in situ green fluorescent protein (GFP) expression by fluorescence imaging. Specifically, HEK293T cells were transiently co-transfected with LID genes to control the transcription of a chromosomally integrated GFP gene. Imaging analysis showed zero to very low GFP expression in cells kept in the dark (FIG. 6a). In LDB-3- and LDB-14-expressing cells, light-induced GFP levels were close and, compared with the expression in the dark, increased by ~44 and ~39 folds, respectively (FIG. 6b).

### CONCLUSIONS

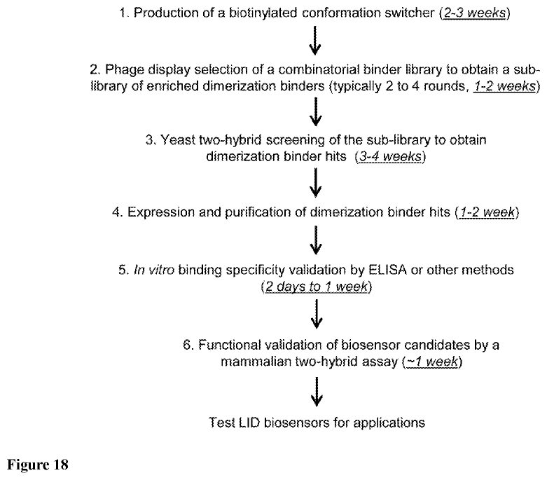

Our work demonstrated that COMBINES-LID is efficient for creating LID systems. This method screened a generic combinatorial nanobody library using fast and cost-effective phage display and Y2H techniques to obtain high-quality, mammalian-applicable binders without need for further engineering of binding affinity and specificity, thus offering a short turnaround time (FIG. 18). It relies on using protein targets with photo-induced light or dark-state specific conformational changes to select binder specificity, and theoretically can be applied to a large array of photoswitchable proteins (Table 2) to create orthogonal LID systems with diverse optical and structural properties. Applicable dimerization binders can also include those with other scaffolds such as non-immunoglobulin²⁸ and computationally designed scaffolds.

The LDB-3 and LDB-14-DrBphP LID systems, now named ‘nanoReD1’ and ‘nanoReD2’, respectively, have simplified structures and improved in vivo performance, overcoming the intrinsic limitations of naturally occurring BphP LID and its derivatives. These systems have been tested for light-activated gene expression, and are also useful for controlling other cellular processes, for example, the spatiotemporal activation of chimeric antigen receptor T (CAR-T) cells. Their use of the mammalian endogenous metabolite as chromophore and the compatibility of deep tissue penetration offer the unique potential to address clinical challenges such as CAR-T therapy targeting solid tumors.

### Abbreviations

LID, light-induced protein dimerization; CID, chemically induced dimerization; COMBINES-LID, combinatorial binders-enabled selection of LID; nanoReD, nanobody-based, red light-induced dimerization; LOV, light-oxygen-voltage; NIR, near-infrared; BphP, bacterial phytochrome; CDR, complementarity-determining region; Y2H, yeast two-hybrid; ELISA, enzyme-linked immunosorbent assay; HEK293T, human embryonic kidney 293T cell; PPI, protein-protein interaction; BD, DNA binding domain; AD, activation domain; SDS-PAGE, sodium dodecyl sulfate gel electrophoresis; SEC, size-exclusion chromatography; ITC, isothermal titration calorimetry; K_{D}^{app}, apparent dissociation constant; BLI, Bio-Layer Interferometry; GFP, green fluorescent protein; CAR-T, chimeric antigen receptor T cell.

### REFERENCES

(1) Zhang, F.; Vierock, J.; Yizhar, O.; Fenno, L. E.; Tsunoda, S.; Kianianmomeni, A.; Prigge, M.; Bemdt, A.; Cushman, J.; Polle, J.; Magnuson, J.; Hegemann, P.; Deisseroth, K. The Microbial Opsin Family of Optogenetic Tools. Cell 2011, 147, 1446-1457.
(2) Mayer, G.; Heckel, A. Biologically active molecules with a “light switch”. Angew. Chem. Int. Ed. Engl. 2006, 45, 4900-4921.
(3) Christie, J. M.; Salomon, M.; Nozue, K.; Wada, M.; Briggs, W. R. LOV (light, oxygen, or voltage) domains of the blue-light photoreceptor phototropin (nphl): Binding sites for the chromophore flavin mononucleotide. Proc. Natl. Acad. Sci. U.S.A 1999, 96, 8779-8783.
(4) Bhoo, S. H.; Davis, S. J.; Walker, J.; Kamiol, B.; Vierstra, R. D. Bacteriophytochromes are photochromic histidine kinases using a biliverdin chromophore. Nature 2001, 414, 776-779.
(5) Rockwell, N. C.; Su, Y. S.; Lagarias, J. C. Phytochrome structure and signaling mechanisms. Annu. Rev. Plant Biol. 2006, 57, 837-858.
(6) Weissleder, R. A clearer vision for in vivo imaging. Nat. Biotechnol. 2001, 19, 316-317.
(7) Weissleder, R.; Ntziachristos, V. Shedding light onto live molecular targets. Nat. Med. 2003, 9, 123-128.
(8) Jobsis, F. F. Noninvasive, infrared monitoring of cerebral and myocardial oxygen sufficiency and circulatory parameters. Science 1977, 198, 1264-1267.
(9) Chemov, K. G.; Redchuk, T. A.; Omelina, E. S.; Verkhushaa, V. V. Near-infrared fluorescent proteins, biosensors, and optogenetic tools engineered from phytochromes. Chem. Rev. 2017, 117, 6423-6446.
(10) Bellini, D.; Papiz, M. Z. Structure of a bacteriophytochrome and light-stimulated protomer swapping with a gene repressor. Structure 2012, 20, 1436-1446.
(11) Winkler, A.; Heintz, U.; Lindner, R.; Reinstein, J.; Shoeman, R. L.; Schlichting, I. A ternary AppA-PpsR-DNA complex mediates light regulation of photosynthesis-related gene expression. Nat. Struct. Mol. Biol. 2013, 20, 859-867.
(12) Kabemiuk, A. A.; Shemetov, A. A.; Verkhusha, V. V. A bacterial phytochrome-based optogenetic system controllable with near-infrared light. Nat. Methods 2016, 13, 591-597.
(13) Redchuk, T. A.; Omelina, E. S.; Chemov, K. G.; Verkhusha, V. V. Near-infrared optogenetic pair for protein regulation and spectral multiplexing. Nat. Chem. Biol. 2017, 13, 633-639.
(14) Kojadinovic, M.; Laugraud, A.; Vuillet, L.; Fardoux, J.; Hannibal, L.; Adriano, J. M.; Bouyer, P.; Giraud, E.; Vermeglio, A. Dual role for a bacteriophytochrome in the bioenergetic control of Rhodopseudomonas palustris: Enhancement of photosystem synthesis and limitation of respiration. Biochim. Biophys. Acta 2008, 1777, 163-172.
(15) Wagner, J. R.; Zhang, J. R.; Brunzelle, J. S.; Vierstra, R. D.; Forest, K. T. High resolution structure of Deinococcus bacteriophytochrome yields new insights into phytochrome architecture and evolution. J Biol. Chem. 2007, 282, 12298-12309.
(16) Yang, X.; Kuk, J.; Moffat, K. Crystal structure of Pseudomonas aeruginosa bacteriophytochrome: Photoconversion and signal transduction. Proc. Natl. Acad. Sci. U.S.A. 2008, 105, 14715-14720.
(17) Guntas, G.; Hallett, R. A.; Zimmerman, S. P.; Williams, T.; Yumerefendi, H.; Bear, J. E.; Kuhlman, B. Engineering an improved light-induced dimer (iLID) for controlling the localization and activity of signaling proteins. Proc. Natl. Acad. Sci. U.S.A 2015, 112, 112-117.
(18) Reis, J. M.; Xu, X. L.; McDonald, S.; Woloschuk, R. M.; Jaikaran, A. S. I.; Vizeacoumar, F. S.; Woolley, G. A.; Uppalapati, M. Discovering selective binders for photoswitchable proteins using phage display. ACS Synth. Biol. 2018, 7, 2355-2364.
(19) Muyldermans, S. Nanobodies: natural single-domain antibodies. Annu. Rev. Biochem. 2013, 82, 775-797.
(20) Kang, S.; Davidsen, K.; Gomez-Castillo, L.; Jiang, H.; Fu, X.; Li, Z.; Liang, Y.; Jahn, M.; Moussa, M.; DiMaio, F.; Gu, L. COMBINES-CID: An efficient method for de novo engineering of highly specific chemically induced protein dimerization systems. J Am. Chem. Soc. 2019, 141, 10948-10952.
(21) Moutel, S.; Bery, N.; Bernard, V.; Keller, L.; Lemesre, E.; de Marco, A.; Ligat, L.; Rain, J. C.; Favre, G.; Olichon, A.; Perez, F. NaLi-H1: A universal synthetic library of humanized nanobodies providing highly functional antibodies and intrabodies. eLife 2016, 5, e16228.
(22) Wagner, J. R.; Zhang, J. R.; von Stetten, D.; Guenther, M.; Murgida, D. H.; Mroginski, M. A.; Walker, J. M.; Forest, K. T.; Hildebrandt, P.; Vierstra, R. D. Mutational analysis of Deinococcus radiodurans bacteriophytochrome reveals key amino acids necessary for the photochromicity and proton exchange cycle of phytochromes. J. Biol. Chem. 2008, 283, 12212-12226.
(23) Takala, H.; Bjorling, A.; Berntsson, O.; Lehtivuori, H.; Niebling, S.; Hoemke, M.; Kosheleva, I.; Henning, R.; Menzel, A.; Ihalainen, J. A.; Westenhoff, S. Signal amplification and transduction in phytochrome photosensors. Nature 2014, 509, 245-248.
(24) Choi, S. G.; Olivet, J.; Cassonnet, P.; Vidalain, P. O.; Lucks, K.; Lambourne, L.; Spirohn, K.; Lemmens, I.; Dos Santos, M.; Demeret, C.; Jones, L.; Rangarajan, S.; Bian, W.; Coutant, E. P.; Janin, Y. L.; van Der Werf, S.; Trepte, P.; Wanke, E. E.; De Las Rivas, J.; Tavernier, J.; Twizere, J. C.; Hao, T.; Hill, D. E.; Vidal, M.; Calderwood, M. A.; Jacob, Y. Maximizing binary interactome mapping with a minimal number of assays. Nat. Commun. 2019, 10, 3907.
(25) Lievens, S.; Lemmens, I.; Tavernier, J. Mammalian two-hybrids come of age. Trends Biochem. Sci. 2009, 34, 579-588.
(26) Ong, N. T.; Olson, E. J.; Tabor, J. J. Engineering an E. coli near-infrared light sensor. ACS Synth. Biol. 2018, 7, 240-248.
(27) Niu, J.; Ben Johny, M.; Dick, I. E.; Inoue, T. Following optogenetic dimerizers and quantitative prospects. Biophys. J. 2016, 111, 1132-1140.
(28) Skrlec, K.; Strukelj, B.; Berlec, A. Non-immunoglobulin scaffolds: a focus on their targets. Trends Biotechnol. 2015, 33, 408-418.
(29) Chevalier, A.; Silva, D. A.; Rocklin, G. J.; Hicks, D. R.; Vergara, R.; Murapa, P.; Bernard, S. M.; Zhang, L.; Lam, K. H.; Yao, G.; Bahl, C. D.; Miyashita, S. I.; Goreshnik, I.; Fuller, J. T.; Koday, M. T.; Jenkins, C. M.; Colvin, T.; Carter, L.; Bohn, A.; Bryan, C. M.; Femindez-Velasco, D. A.; Stewart, L.; Dong, M.; Huang, X.; Jin, R.; Wilson, I. A.; Fuller, D. H.; Baker, D. Massively parallel de novo protein design for targeted therapeutics. Nature 2017, 550, 74-79.
(30) Wu, C. Y.; Roybal, K. T.; Puchner, E. M.; Onuffer, J.; Lim, W. A. Remote control of therapeutic T cells through a small molecule-gated chimeric receptor. Science 2015, 350, aab4077.
(31) Huang, Z. L.; Wu, Y. Q.; Allen, M. E.; Pan, Y. J.; Kyriakakis, P.; Lu, S. Y.; Chang, Y. J.; Wang, X.; Chien, S.; Wang, Y. X. Engineering light-controllable CAR T cells for cancer immunotherapy. Sci. Adv. 2020, eaay9209

### Supplementary Methods

### Plasmid Construction

Primers and protein coding sequence (CDSs) for plasmid construction were synthesized by Integrated DNA Technologies (IDT) or amplified from other plasmids. CDSs, noncommercial plasmid sequences, and subcloning insertion sites are listed in Table 7. The subcloning was performed using a Gibson assembly protocol recently described.

TABLE 7
Protein coding sequences (CDSs) and
noncommercial vector used in this work.

| | | | Subcloning |
|---|---|---|---|
| Purpose | Name | CDS or vector sequence | note |
| E. coli | DrBp | ATGAGTCGTGACCCTTTGCCATTCTTTCCTCCTCTTTATC | The CDS was |
| expression | hP- | TGGGTGGACCCGAGATTACAACAGAAAACTGCGAACGCGA | inserted into |
| | Avi- | ACCAATTCACATCCCGGGATCTATTCAACCACACGGTGCA | pBAD (Addgene |
| | His | TTGCTGACGGCAGACGGACATTCCGGAGAGGTTTTACAGA | #80341) using |
| | | TGTCGCTTAACGCAGCAACGTTTCTGGGACAAGAGCCTAC | BamHI/EcoRI |
| | | GGTTTTGCGCGGCCAGACGTTAGCGGCTCTGTTGCCAGAG | restriction sites. |
| | | CAATGGCCGGCCTTACAGGCGGCATTGCCTCCAGGGTGCC | |
| | | CCGATGCATTGCAATACCGCGCGACACTGGATTGGCCGGC | |
| | | GGCAGGACATCTTTCTCTGACAGTCCACCGCGTGGGCGAG | |
| | | CTGTTGATCCTGGAGTTTGAACCTACGGAGGCCTGGGACT | |
| | | CGACTGGCCCGCACGCGTTACGCAATGCGATGTTCGCTCT | |
| | | TGAATCAGCGCCAAACTTGCGCGCGTTAGCTGAAGTGGCC | |
| | | ACACAAACCGTACGCGAGCTTACAGGCTTTGACCGCGTGA | |
| | | TGTTATACAAATTCGCACCCGATGCGACAGGCGAGGTAAT | |
| | | CGCCGAAGCCCGCCGCGAGGGGTTGCATGCCTTTCTTGGC | |
| | | CATCGTTTTCCGGCCTCAGATATTCCCGCCCAAGCGCGCG | |
| | | CCCTTTACACTCGCCATCTGCTTCGTTTGACTGCGGACAC | |
| | | GCGCGCGGCGGCCGTTCCCTTAGACCCAGTACTTAATCCT | |
| | | CAGACTAACGCTCCTACCCCCTTAGGGGGGGCAGTGCTGC | |
| | | GTGCGACGTCGCCTATGCACATGCAGTACCTTCGCAATAT | |
| | | GGGCGTCGGCTCCTCTTTAAGTGTATCAGTGGTAGTTGGG | |
| | | GGGCAGTTATGGGGTCTGATTGCGTGCCATCATCAGACCC | |
| | | CCTATGTTTTGCCACCAGACCTTCGTACTACTCTTGAATA | |
| | | CTTGGGGCGTTTATTAAGCCTTCAGGTGCAAGTCAAGGAA | |
| | | GCCGCGGACGTTGCTGCATTCCGTCAGTCACTTCGCGAAC | |
| | | ACCATGCGCGCGTCGCCTTAGCGGCAGCGCATTCCCTGTC | |
| | | GCCGCACGATACTCTTTCCGACCCTGCACTTGATCTTCTG | |
| | | GGTCTGATGCGTGCTGGGGGCTTAATCCTGCGTTTTGAAG | |
| | | GTCGTTGGCAGACGTTAGGAGAAGTCCCGCCCGCTCCCGC | |
| | | AGTCGATGCACTGCTTGCATGGCTTGAAACCCAACCAGGG | |
| | | GCGCTTGTTCAGACTGATGCATTGGGGCAGTTGTGGCCGG | |
| | | CGGGGGCTGATTTGGCTCCCTCAGCCGCGGGTCTGCTTGC | |
| | | CATTTCAGTAGGGGAGGGATGGAGTGAGTGCTTGGTTTGG | |
| | | TTACGTCCCGAACTGCGCCTTGAGGTTGCGTGGGGTGGAG | |
| | | CAACTCCAGACCAGGCCAAGGACGACCTGGGCCCTCGTCA | |
| | | CAGTTTCGATACTTACTTAGAAGAGAAGCGTGGGTATGCA | |
| | | GAACCCTGGCATCCCGGAGAGATTGAGGAAGCTCAGGATT | |
| | | TGCGCGACACTCTTACTGGCGCATTAAAGCTTGGTGGCGG | |
| | | TAGCGAGAATTTGTATTTTCAGGGTGGCGGTGGCAGTAGC | |
| | | TTATCCACCCCGCCGACCCCGAGCACTCCTCCTACCGGTC | |
| | | TGAACGACATCTTCGAGGCTCAGAAAATCGAATGGCACGA | |
| | | ACATCATCACCACCATCAC (SEQ ID NO: 21) | |
| | DrBp | (DrBphP)-GAATTCCATCATCACCATCACCAT (SEQ | The sequence |
| | hP- | ID NO: 22) | of DrBphP is |
| | His | | the same as |
| | | | above. The |
| | | | CDS was |
| | | | inserted into |
| | | | pBAD (Addgene |
| | | | #80341) using |
| | | | BamHI/EcoRI |
| | | | restriction sites. |
| | LDB- | GAAGTTCAGCTGCAGGCAAGCGGTGGTGGTTTTGTTCAGC | The CDS was |
| | 3-His | CTGGTGGTAGCCTGCGTCTGAGCTGTGCAGCCAGCGGTTT | inserted into |
| | | TACCTGGGATCATTACATCATGGGCTGGTTTCGCCAGGCA | pADL-23c |
| | | CCGGGTAAAGAACGTGAATTTGTTAGCGCAATCAGCGAAA | (Antibody |
| | | ATGGTGATGCATGGAATTATTATGCCGATAGCGTGAAAGG | Design Labs) |
| | | TCGCTTTACCATTAGCCGTGATAATAGCAAAAATACCGTT | using a BgII |
| | | TACCTGCAGATGAATAGTCTGCGTGCAGAAGATACCGCAA | restriction site. |
| | | CCTATTATTGTGCAATCGGTTTTGATGTTCCATCTGGTCG | |
| | | TTCTTGGCAGGGTTCTCATTTTTGGATGTATTGGGGTCAG | |
| | | GGCACCCAGGTTACCGTTAGCAGCAGCCCGGGAGGCCAAC | |
| | | ACCATCACCACCATCAT (SEQ ID NO: 23) | |
| | LDB- | (LDB-3)- | The sequence |
| | 3-Avi- | AGCCCGGGAGGCCAAAGCTTATCCACCCCGAGTGTAGATC | of LDB-3 is the |
| | His | TCGGTGGTCGCCGTATCATTGGTCTGAACGACATCTTCGA | same as above. |
| | | GGCTCAGAAAATCGAATGGCACGAACATCATCACCACCAT | The CDS was |
| | | CACTCT (SEQ ID NO: 24) | inserted into |
| | | | pADL-23c using |
| | | | a BgII restriction |
| | | | site. |
| | LDB- | GAAGTTCAGCTGCAGGCAAGCGGTGGTGGTTTTGTTCAGC | The CDS was |
| | 14- | CTGGTGGTAGCCTGCGTCTGAGCTGTGCAGCCAGCGGTAC | inserted into |
| | His | CACCTCTCGTTGGGAATCTATGGGCTGGTTTCGCCAGGCA | pADL-23c using |
| | | CCGGGTAAAGAACGTGAATTTGTTAGCGCAATCAGCTGGC | a BgII restriction |
| | | AGAATAATTCTGTTCCATATTATGCCGATAGCGTGAAAGG | site. |
| | | TCGCTTTACCATTAGCCGTGATAATAGCAAAAATACCGTT | |
| | | TACCTGCAGATGAATAGTCTGCGTGCAGAAGATACCGCAA | |
| | | CCTATTATTGTGCAGCACAGCATAACTTTCTGGGTCATCG | |
| | | TTATTGGGGTCAGGGCACCCAGGTTACCGTTAGCAGCAGC | |
| | | CCGGGAGGCCAACACCATCACCACCATCAT (SEQ ID | |
| | | NO: 25) | |
| | LDB- | (LDB-14)- | The sequence |
| | 14- | AGCCCGGGAGGCCAAAGCTTATCCACCCCGAGTGTAGATC | of LDB-14 is the |
| | Avi- | TCGGTGGTCGCCGTATCATTGGTCTGAACGACATCTTCGA | same as above. |
| | His | GGCTCAGAAAATCGAATGGCACGAACATCATCACCACCAT | The CDS was |
| | | CACTCT (SEQ ID NO: 26) | inserted into |
| | | | pADL-23c using |
| | | | a BgII restriction |
| | | | site. |
| Yeast | GAL4- | ATGAAGCTACTGTCTTCTATCGAACAAGCATGCGATATTT | The sequence |
| two- | DrBp | GCCGACTTAAAAAGCTCAAGTGCTCCAAAGAAAAACCGAA | of DrBphP is |
| hybrid | hP | GTGCGCCAAGTGTCTGAAGAACAACTGGGAGTGTCGCTAC | the same as |
| | | TCTCCCAAAACCAAAAGGTCTCCGCTGACTAGGGCACATC | above. The |
| | | TGACAGAAGTGGAATCAAGGCTAGAAAGACTGGAACAGCT | CDS was |
| | | ATTTCTACTGATTTTTCCTCGAGkAGACCTTGACATGATT | inserted into |
| | | TTGAAAATGGATTCTTTACAGGATATAAAAGCATTGTTAA | pGBKT7 vector |
| | | CAGGATTATTTGTACAAGATAATGTGAATAAAGATGCCGT | (Clontech) |
| | | CACAGATAGATTGGCTTCAGTGGAGACTGATATGCCTCTA | using |
| | | ACATTGAGACAGCATAGAATAAGTGCGACATCATCATCGG | NdeI/BamHI |
| | | AAGAGAGTAGTAACAAAGGTCAAAGACAGTTGACTGTATC | restriction sites. |
| | | GCCGGAATTTGTAATACGACTCACTATAGGGCGAGCCGCC | |
| | | ATCATGGAGGAGCAGAAGCTGATCTCAGAGGAGGACCTGC | |
| | | AT-(DrBphP) (SEQ ID NO: 27) | |
| | AD- | ATGGATAAAGCGGAATTAATTCCCGAGCCTCCAAAAAAGA | The sequence |
| | LDB- | AGAGAAAGGTCGAATTGGGTACCGCCGCCAATTTTAATCA | of LDB-3 is the |
| | 3 | AAGTGGGAATATTGCTGATAGCTCATTGTCCTTCACTTTC | same as above. |
| | | ACTAACAGTAGCAACGGTCCGAACCTCATAACAACTCAAA | The CDS was |
| | | CAAATTCTCAAGCGCTTTCACAACCAATTGCCTCCTCTAA | inserted into |
| | | CGTTCATGATAACTTCATGAATAATGAAATCACGGCTAGT | pGADT7 |
| | | AAAATTGATGATGGTAATAATTCAAAACCACTGTCACCTG | (Clontech) |
| | | GTTGGACGGACCAAACTGCGTATAACGCGTTTGGAATCAC | using |
| | | TACAGGGATGTTTAATACCACTACAATGGATGATGTATAT | NdeI/BamHI |
| | | AACTATCTATTCGATGATGAAGATACCCCACCAAACCCAA | restriction sites. |
| | | AAAAAGAGATCTTTAATACGACTCACTATAGGGCGAGCGC | |
| | | CGCCATGGAGTACCCATACGACGTACCAGATTACGCTCAT | |
| | | ATGGGAGGCGGTTCCGGTGGCGGTTCT-(LDB-3) (SEQ | |
| | | ID NO: 28) | |
| | AD- | (AD)- | The CDS was |
| | LDB- | ATCTTTAATACGACTCACTATAGGGCGAGCGCCGCCATGG | inserted into |
| | 4 | AGTACCCATACGACGTACCAGATTACGCTCATATGGGAGG | pGADT7 using |
| | | CGGTTCCGGTGGCGGTTCTGAAGTTCAGCTGCAGGCAAGC | NdeI/BamHI |
| | | GGTGGTGGTTTTGTTCAGCCTGGTGGTAGCCTGCGTCTGA | restriction sites. |
| | | GCTGTGCAGCCAGCGGTGATACCTCTTACCTGTACTCTAT | The sequences |
| | | GGGCTGGTTTCGCCAGGCACCGGGTAAAGAACGTGAATTT | of AD and LDB- |
| | | GTTAGCGCAATCAGCTGGTGGTGGAATCTGACTCAGTATT | 14 are the same |
| | | ATGCCGATAGCGTGAAAGGTCGCTTTACCATTAGCCGTGA | as above. |
| | | TAATAGCAAAAATACCGTTTACCTGCAGATGAATAGTCTG | |
| | | CGTGCAGAAGATACCGCAACCTATTATTGTGCATGGTCTA | |
| | | TCTACTTTCCACCAGGTAACGATTACAACGGTTACCATTA | |
| | | TTGGGGTCAGGGCACCCAGGTTACCGTTAGCAGC(SEQ | |
| | | ID NO: 29) | |
| | AD- | (AD)- | |
| | LDB- | ATCTTTAATACGACTCACTATAGGGCGAGCGCCGCCATGG | |
| | 6 | AGTACCCATACGACGTACCAGATTACGCTCATATGGGAGG | |
| | | CGGTTCCGGTGGCGGTTCTGAAGTTCAGCTGCAGGCAAGC | |
| | | GGTGGTGGTTTTGTTCAGCCTGGTGGTAGCCTGCGTCTGA | |
| | | GCTGTGCAGCCAGCGGTTTTTTTTCTAACTGGTCTGATAT | |
| | | GGGCTGGTTTCGCCAGGCACCGGGTAAAGAACGTGAATTT | |
| | | GTTAGCGCAATCAGCTTTTGGGCAGATGGTACTGAATATT | |
| | | ATGCCGATAGCGTGAAAGGTCGCTTTACCATTAGCCGTGA | |
| | | TAATAGCAAAAATACCGTTTACCTGCAGATGAATAGTCTG | |
| | | CGTGCAGAAGATACCGCAACCTATTATTGTGCATGGTACG | |
| | | GTCCAGTTAACGGTTTTTACATGTTTGATTATTGGGGTCA | |
| | | GGGCACCCAGGTTACCGTTAGCAGC(SEQ ID NO: 30) | |
| | AD- | (AD)- | |
| | LDB- | ATCTTTAATACGACTCACTATAGGGCGAGCGCCGCCATGG | |
| | 7 | AGTACCCATACGACGTACCAGATTACGCTCATATGGGAGG | |
| | | CGGTTCCGGTGGCGGTTCTGAAGTTCAGCTGCAGGCAAGC | |
| | | GGTGGTGGTTTTGTTCAGCCTGGTGGTAGCCTGCGTCTGA | |
| | | GCTGTGCAGCCAGCGGTTCTACCTCTGATTTTGAATCTAT | |
| | | GGGCTGGTTTCGCCAGGCACCGGGTAAAGAACGTGAATTT | |
| | | GTTAGCGCAATCAGCTCTTGGTTTACTAATCCACCATATT | |
| | | ATGCCGATAGCGTGAAAGGTCGCTTTACCATTAGCCGTGA | |
| | | TAATAGCAAAAATACCGTTTACCTGCAGATGAATAGTCTG | |
| | | CGTGCAGAAGATACCGCAACCTATTATTGTGCACATCGTT | |
| | | CTATCTGGTACCATCCAACCTATTGGGGTCAGGGCACCCA | |
| | | GGTTACCGTTAGCAGC(SEQ ID NO: 31) | |
| | AD- | (AD)- | |
| | LDB- | ATCTTTAATACGACTCACTATAGGGCGAGCGCCGCCATGG | |
| | 14 | AGTACCCATACGACGTACCAGATTACGCTCATATGGGAGG | |
| | | CGGTTCCGGTGGCGGTTCT-(LDB-14) (SEQ ID NO: | |
| | | 32) | |
| Mammalian | GAL4 | GAL4-DrBphP | The sequence |
| two- | DrBp | | ofGAL4- |
| hybrid | hP | | DrBphP is the |
| | | | same as above. |
| | | | The CDS was |
| | | | inserted into |
| | | | pBobi (see |
| | | | below for the |
| | | | sequence) |
| | | | using |
| | | | BamHI/XhoI |
| | | | restriction sites. |
| | NLS- | ATGGGATCCCCCAAGAAGAAGCGCAAGGTGGAAGCTAGCG | The CDS was |
| | LDB- | CTTCCCCGAAGAAAAAGCGGAAAGTCGAGGCCTCCGCATC | inserted into |
| | 3-p65 | TCCAAAAAAAAAAAGCAAGGTTGAAGCATCTGGATCCGGT | pBobi vector |
| | | ACCGGAGGAAGTGGCAGCTCTGGCGGCAGTGGAGGGTCTG | using |
| | | GTGGCAGCGGA-(LDB-3)- | BamHI/XhoI |
| | | AGCGGCGGCGGTGGCAGTCAGTACCTGCCAGATACAGACG | restriction sites. |
| | | ATCGTCACCGGATTGAGGAGAAACGTAAAAGGACATATGA | The sequence |
| | | GACCTTCAAGAGCATCATGAAGAAGAGTCCTTTCAGCGGA | of LDB-3 is the |
| | | CCCACCGACCCCCGGCCTCCACCTCGACGCATTGCTGTGC | same as above. |
| | | CTTCCCGCAGCTCAGCTTCTGTCCCCAAGCCAGCACCCCA | |
| | | GCCCTATCCCTTTACGTCATCCCTGAGCACCATCAACTAT | |
| | | GATGAGTTTCCCACCATGGTGTTTCCTTCTGGGCAGATCA | |
| | | GCCAGGCCTCGGCCTTGGCCCCGGCCCCTCCCCAAGTCCT | |
| | | GCCCCAGGCTCCAGCCCCTGCCCCTGCTCCAGCCATGGTA | |
| | | TCAGCTCTGGCCCAGGCCCCAGCCCCTGTCCCAGTCCTAG | |
| | | CCCCAGGCCCTCCTCAGGCTGTGGCCCCACCTGCCCCCAA | |
| | | GCCCACCCAGGCTGGGGAAGGAACGCTGTCAGAGGCCCTG | |
| | | CTGCAGCTGCAGTTTGATGATGAAGACCTGGGGGCCTTGC | |
| | | TTGGCAACAGCACAGACCCAGCTGTGTTCACAGACCTGGC | |
| | | ATCCGTCGACAACTCCGAGTTTCAGCAGCTGCTGAACCAG | |
| | | GGCATACCTGTGGCCCCCCACACAACTGAGCCCATGCTGA | |
| | | TGGAGTACCCTGAGGCTATAACTCGCCTAGTGACAGGGGC | |
| | | CCAGAGGCCCCCCGACCCAGCTCCTGCTCCACTGGGGGCC | |
| | | CCGGGGCTCCCCAATGGCCTCCTTTCAGGAGATGAAGACT | |
| | | TCTCCTCCATTGCGGACATGGACTTCTCAGCCCTGCTGAG | |
| | | TCAGATCAGCTCC(SEQ ID NO: 33) | |
| | NLS- | (NLS)- | The sequences |
| | LDB- | GGATCCGGTACCGGAGGAAGTGGCAGCTCTGGCGGCAGTG | of NLS, |
| | 4-p65 | GAGGGTCTGGTGGCAGCGGA-(LDB-4)- | nanobody and |
| | | AGCGGCGGCGGTGGCAGT-(p65) (SEQ ID NO: 34) | p65 are the |
| | | | same as above. |
| | | | The CDS was |
| | | | inserted into |
| | | | pBobi using |
| | | | BamHI/XhoI |
| | | | restriction sites. |
| | NLS- | (NLS)- | |
| | LDB- | GGATCCGGTACCGGAGGAAGTGGCAGCTCTGGCGGCAGTG | |
| | 6-p65 | GAGGGTCTGGTGGCAGCGGA-(LDB-6)- | |
| | | AGCGGCGGCGGTGGCAGT-(p65) (SEQ ID NO: 35) | |
| | NLS- | (NLS)- | |
| | LDB- | GGATCCGGTACCGGAGGAAGTGGCAGCTCTGGCGGCAGTG | |
| | 7-p65 | GAGGGTCTGGTGGCAGCGGA-(LDB-7)- | |
| | | AGCGGCGGCGGTGGCAGT-(p65) (SEQ ID NO: 36) | |
| | NLS- | ATGCCCAAGAAGAAGCGCAAGGTGGAAGCTAGCGCTTCCC | The CDS was |
| | LDB- | CGAAGAAAAAGCGGAAAGTCGAGGCCTCCGCATCTCCAAA | inserted into |
| | 14- | AAAAAAAAGCAAGGTTGAAGCATCTGGATCCGGAGGCGGT | pcDNA3 |
| | p65 | TCCGGTGGCGGTTCT-(LDB-14)- | (Invitrogen) |
| | | GGTACCGGAGGAAGTGGCAGCTCTGGCGGCAGTGGAGGGT | using |
| | | CTGGTGGCAGCGGA-(p65)- | HindIII/XhoI |
| | | GGAGGAAGTGGCAGCTCTGGCGGCAGTGGA(SEQ ID | restriction sites. |
| | | NO: 37) | The sequences |
| | | | of LDB-14 and |
| | | | p65 are the |
| | | | same as above. |
| | GAL4 | (GAL4)- | The CDS was |
| | RpBp | CCGGAATTTGTAATACGACTCACTATAGGGCGAGCCGCCA | subcloned into |
| | hP1 | TCATGGAGGAGCAGAAGCTGATCTCAGAGGAGGACCTGCA | pcDNA3 using |
| | | TGTGGCAGGTCATGCCTCTGGCAGCCCCGCATTCGGGACC | HindIII/XhoI |
| | | GCCGATCTTTCGAATTGCGAACGTGAAGAGATCCACCTCG | restriction sites. |
| | | CCGGCTCGATCCAGCCGCATGGCGCGCTTCTGGTCGTCAG | The sequence |
| | | CGAGCCGGATCATCGCATCATCCAGGCCAGCGCCAACGCC | ofGAL4 is the |
| | | GCGGAATTTCTGAATCTCGGAAGCGTGCTCGGCGTTCCGC | same as above. |
| | | TCGCCGAGATCGACGGCGATCTGTTGATCAAGATCCTGCC | |
| | | GCATCTCGATCCCACCGCCGAAGGCATGCCGGTCGCGGTG | |
| | | CGCTGCCGGATCGGCAATCCCTCCACGGAGTACGACGGTC | |
| | | TGATGCATCGGCCTCCGGAAGGCGGGCTGATCATCGAGCT | |
| | | CGAACGTGCCGGCCCGCCGATCGATCTGTCCGGCACGCTG | |
| | | GCGCCGGCGCTGGAGCGGATCCGCACGGCGGGCTCGCTGC | |
| | | GCGCGCTGTGCGATGACACCGCGCTGCTGTTTCAGCAGTG | |
| | | CACCGGCTACGACCGGGTGATGGTGTATCGCTTCGACGAG | |
| | | CAGGGCCACGGCGAAGTGTTCTCCGAGCGCCACGTGCCCG | |
| | | GGCTCGAATCCTATTTCGGCAACCGCTATCCGTCGTCGGA | |
| | | CATTCCGCAGATGGCGCGGCGGCTGTACGAGCGGCAGCGC | |
| | | GTCCGCGTGCTGGTCGACGTCAGCTATCAGCCGGTGCCGC | |
| | | TGGAGCCGCGGCTGTCGCCGCTGACCGGGCGCGATCTCGA | |
| | | CATGTCGGGCTGCTTCCTGCGCTCGATGTCGCCGATCCAT | |
| | | CTGCAGTACCTGAAGAACATGGGCGTGCGCGCCACCCTGG | |
| | | TGGTGTCGCTGGTGGTCGGCGGCAAGCTGTGGGGCCTGGT | |
| | | TGCCTGTCACCATTATCTGCCGCGCTTCATCCATTTCGAG | |
| | | CTGCGGGCGATCTGCGAACTGCTCGCCGAAGCGATCGCGA | |
| | | CGCGGATCACCGCGCTTGAGAGCTTCGCGCAGAGCCAGTC | |
| | | GGAGCTGTTCGTGCAGCGGCTCGAACAGCGCATGATCGAA | |
| | | GCGATCACCCGTGAAGGCGATTGGCGCGCAGCGATTTTCG | |
| | | ACACCAGCCAATCGATCCTGCAGCCGCTGCACGCCGACGG | |
| | | TTGCGCGCTGGTGTACGAAGACCAGATCAGGACCATCGGT | |
| | | GACGTACCTTCCACGCAGGATGTTCGCGAGATCGCCGGGT | |
| | | GGCTCGATCGCCAGCCACGTGCGGCGGTGACCTCGACCGC | |
| | | GTCGCTCGGTCTCGACGTGCCGGAGCTCGCGCATCTGACG | |
| | | CGGATGGCGAGCGGCGTGGTCGCGGCGCCGATTTCGGATC | |
| | | ATCGCGGCGAGTTTCTGATGTGGTTCCGCCCCGAGCGCGT | |
| | | CCACACCGTTACCTGGGGCGGCGATCCGAAGAAGCCGTTC | |
| | | ACGATGGGCGATACACCGGCGGATCTGTCGCCGCGGCGCT | |
| | | CCTTCGCCAAATGGCATCAGGTTGTCGAAGGCACGTCCGA | |
| | | TCCGTGGACGGCCGCCGATCTCGCCGCGGCTCGCACCATC | |
| | | GGTCAGACCGTCGCCGACATCGTGCTGCAATTCCGCGCGG | |
| | | TGCGGACACTGATCGCCCGCGAACAGTACGAACAGTTTTC | |
| | | GTCCCAGGTGCACGCTTCGATGCAGCCGGTGCTGATCACC | |
| | | GACGCCGAAGGCCGCATCCTGCTGATGAACGACTCGTTCC | |
| | | GCGACATGTTGCCGGCGGGGTCGCCATCCGCCGTCCATCT | |
| | | CGACGATCTCGCCGGGTTCTTCGTCGAATCGAACGATTTC | |
| | | CTGCGCAACGTCGCCGAACTGATCGATCACGGCCGCGGGT | |
| | | GGCGCGGCGAAGTTCTGCTGCGCGGCGCAGGTAATCGCCC | |
| | | GTTGCCGCTGGCAGTGCGCGCCGATCCGGTGACGCGCACG | |
| | | GAGGACCAGTCGCTCGGCTTCGTGCTGATCTTCAGCGACG | |
| | | CTACCGATCGTCGCACCGCAGATGCCGCACGCACGCGTTT | |
| | | CCAGGAAGGCATTCTTGCCAGCGCACGTCCCGGCGTGCGG | |
| | | CTCGACTCCAAGTCCGACCTCTTGCACGAGAAGCTGCTGT | |
| | | CCGCGCTGGTCGAGAACGCGCAGCTTGCCGCATTGGAAAT | |
| | | TACTTACGGCGTCGAGACCGGACGCATCGCCGAGCTGCTC | |
| | | GAAGGCGTTCGCCAGTCGATGCTGCGCACCGCCGAAGTGC | |
| | | TCGGCCATCTGGTGCAGCACGCGGCGCGCACGGCCGGCAG | |
| | | CGACAGCTCGAGCAATGGCTCGCAGAACAAGAAG | |
| | | (SEQ ID NO: 38) | |
| | NLS- | ATGCCCAAGAAGAAGCGCAAGGTGGAAGCTAGCGCTTCCC | The CDSs were |
| | PpsR | CGAAGAAAAAGCGGAAAGTCGAGGCCTCCGCATCTCCAAA | inserted into |
| | 2-p65 | AAAAAAAAGCAAGGTTGAAGCATCTGGATCCGGAGGCGGT | pcDNA3 using |
| | | TCCGGTGGCGGTTCTGTGGCGTCAAAGTCCGTTCATGCCG | HindIII/XhoI |
| | | ACATCACCCTTCTGCTCGATATGGAGGGTGTGATTCGCGA | restriction sites. |
| | | AGCCACCCTGTCTCCGACGATGGCGGCCGAGAGCGTGGAC | The sequence |
| | | GGTTGGCTGGGGCGTCGCTGGAGCGACATCGCCGGCGCCG | of p65 is the |
| | | AAGGCGGCGACAAGGTTCGCCGCATGGTCGAAGACGCCCG | same as above. |
| | | CCGCAGCGGCATCTCGGCTTTCCGCCAGATCAATCAGCCT | |
| | | TTCCCGAGCGGCGTCGAAATCCCGATCGAATTCACCACGA | |
| | | TGCTGCTGGGCGACCGCACCGGCATGATCGCGGTCGGCAA | |
| | | GAACATGCAGGCGGTCACCGAGCTGCATTCCCGGCTGATC | |
| | | GCTGCGCAGCAGGCGATGGAGCGCGACTATTGGCGGTTGC | |
| | | GTGAATTGGAGACTCGCTACCGCCTGGTGTTCGACGCTGC | |
| | | CGCCGATGCGGTGATGATCGTCTCCGCCGGCGACATGCGC | |
| | | ATCGTCGAAGCCAACCGGGCGGCGGTGAATGCGATCAGCC | |
| | | GCGTCGAGCGCGGCAATGACGACCTTGCGGGGCGTGATTT | |
| | | CCTCGCCGAAGTGGCGGCTGCCGATCGCGATGCGGTGCGC | |
| | | GACATGCTGGCCCAGGTGCGTCAGCGCGGCACCGCACTCA | |
| | | GCGTCCTCGTTCATCTCGGCCGTTACGACCGCGCCTGGAT | |
| | | GCTGCGCGGTTCGCTGATGTCGTCCGAGCGTCGTCAGGTT | |
| | | TTCCTGCTGCACTTCACCCCGGTGACCACGACTCCCGCGA | |
| | | TCGACGACGTCGACGATGATGCCGTGCTGCGCGGGCTGAT | |
| | | CGATCGCATTCCCGACGGGTTCGTCGCACTGGATTCGGAA | |
| | | GGCGTCGTTCGTCACGCCAACCAGGCGTTTCTCGATCTGG | |
| | | TCCAGATCGGCTCCAAGCCTGCGGCGGTCGGACGATCGCT | |
| | | GGGCGTCTGGATGGGTCGTCCGGGCGCCGATCTGTCCAGC | |
| | | TTGCTGACGCTGCTGCGGCGCTACAAGACGGTGCGGCTGT | |
| | | TCCAAACGACGATCCGCGGCGAGCTCGGCACCGAGACTGA | |
| | | AGTCGAGGTCTCGGCCGTCGACGGCGAGGACGACCAATAC | |
| | | ATCGGCGTTCTGATGCGCAATGTCGCGCGACGCCTCGACG | |
| | | CTGCGGACGACCACGATGCCTTGCGTCAGGCGCTCGGCCC | |
| | | GATCAGCAAGCAGCTCGGGCGATCCTCGCTGCGCAAGCTG | |
| | | GTGAAGAACGCCGTGAGCATTGTCGAGCAGCACTACGTGA | |
| | | AGGAAGCGCTGTTGCGATCCAAGGGCAATCGCACGGCAAC | |
| | | TGCCGAACTGCTCGGATTGAGCCGGCAGAGCCTTTATGCA | |
| | | AAACTCAACTCCTACGGCTTCGACGACAAAGGTGTCGTTG | |
| | | CTTCTGCTGCCGACGGTGCAGAGGGCGCCTCAGACGACGC | |
| | | AGAGGATGGTACCGGAGGAAGTGGCAGCTCTGGCGGCAGT | |
| | | GGAGGGTCTGGTGGCAGCGGA-(p65)- | |
| | | GGAGGAAGTGGCAGCTCTGGCGGCAGTGGA (SEQ ID | |
| | | NO: 39) | |
| | NLS- | ATGCCCAAGAAGAAGCGCAAGGTGGAAGCTAGCGCTTCCC | |
| | Q- | CGAAGAAAAAGCGGAAAGTCGAGGCCTCCGCATCTCCAAA | |
| | PAS1 | AAAAAAAAGCAAGGTTGAAGCATCTGGATCCGGAGGCGGT | |
| | -p65 | TCCGGTGGCGGTTCTGGCAAGAACATGCAGGCGGTCACCG | |
| | | AGCTGCATTCCCGGCTGATCGCTGCGCAGCAGGCGATGGA | |
| | | GCGCGACTATTGGCGGTTGCGTGAATTGGAGACTCGCTAC | |
| | | CGCCTGGTGTTCGACGCTGCCGCCGATGCGGTGATGATCG | |
| | | TCTCCGCCGGCGACATGCGCATCGTCGAAGCCAACCGGGC | |
| | | GGCGGTGAATGCGATCAGCCGCGTCGAGCGCGGCAATGAC | |
| | | GACCTTGCGGGGCGTGATTTCCTCGCCGAAGTGGCGGCTG | |
| | | CCGATCGCGATGCGGTGCGCGACATGCTGGCCCAGGTGCG | |
| | | TCAGCGCGGCACCGCACTCAGCGTCCTCGTTCATCTCGGC | |
| | | CGTTACGACCGCGCCTGGATGCTGCGCGGTTCGCTGATGT | |
| | | CGTCCGAGCGTCGTCAGGTTTTCCTGCTGCACTTCACCCC | |
| | | GGTGACCACGACTCCCGCGATCGACGACGGTACCGGAGGA | |
| | | AGTGGCAGCTCTGGCGGCAGTGGAGGGTCTGGTGGCAGCG | |
| | | GA-(p65)-GGAGGAAGTGGCAGCTCTGGCGGCAGTGGA | |
| | | (SEQ ID NO: 40) | |
| Detection | LDB- | ATGGGATCC-(LDB-3)- | The CDS was |
| of | 3- | GTTAACGGCGGCGGTGGCAGTGACAAAGACTGCGAAATGA | inserted into |
| nanobody | SNA | AGCGCACCACCCTGGATAGCCCTCTGGGCAAGCTGGAACT | pBobi vector |
| expression | P | GTCTGGGTGCGAACAGGGCCTGCACCGTATCATCTTCCTG | using |
| in | | GGCAAAGGAACATCTGCCGCCGACGCCGTGGAAGTGCCTG | BamHI/XhoI |
| mammalian | | CCCCAGCCGCCGTGCTGGGCGGACCAGAGCCACTGATGCA | restriction sites. |
| cells | | GGCCACCGCCTGGCTCAACGCCTACTTTCACCAGCCTGAG | The sequence |
| | | GCCATCGAGGAGTTCCCTGTGCCAGCCCTGCACCACCCAG | of LDB-3 is the |
| | | TGTTCCAGCAGGAGAGCTTTACCCGCCAGGTGCTGTGGAA | same as above. |
| | | ACTGCTGAAAGTGGTGAAGTTCGGAGAGGTCATCAGCTAC | |
| | | AGCCACCTGGCCGCCCTGGCCGGCAATCCCGCCGCCACCG | |
| | | CCGCCGTGAAAACCGCCCTGAGCGGAAATCCCGTGCCCAT | |
| | | TCTGATCCCCTGCCACCGGGTGGTGCAGGGCGACCTGGAC | |
| | | GTGGGGGGCTACGAGGGCGGGCTCGCCGTGAAAGAGTGGC | |
| | | TGCTGGCCCACGAGGGCCACAGACTGGGCAAGCCTGGGCT | |
| | | GGGT (SEQ ID NO: 41) | |
| | LDB- | ATGGGATCC-(LDB-4)-GTTAACGGCGGCGGTGGCAGT- | The CDSs were |
| | 4- | (SNAP) (SEQ ID NO: 42) | inserted into |
| | SNA | | pBobi using |
| | P | | BamHI/XhoI |
| | | | restriction sites. |
| | | | The sequences |
| | | | of nanobodies |
| | | | and SNAP are |
| | | | the same as |
| | | | above. |
| | LDB- | ATGGGATCC-(LDB-6)-GTTAACGGCGGCGGTGGCAGT- | |
| | 6- | (SNAP) (SEQ ID NO: 43) | |
| | SNA | | |
| | P | | |
| | LDB- | ATGGGATCC-(LDB-7)-GTTAACGGCGGCGGTGGCAGT- | |
| | 7- | (SNAP) (SEQ ID NO: 44) | |
| | SNA | | |
| | P | | |
| | LDB- | ATGGGATCC-(LDB-14)- | |
| | 14- | GTTAACGGCGGCGGTGGCAGT-(SNAP) (SEQ ID NO: | |
| | SNA | 45) | |
| | P | | |

### Protein Expression and Purification

DrBphP-Avi-His and DrBphP-His were expressed in Escherichia coli and purified by Ni-affinity and size-exclusion chromatography. In brief, Escherichia coli C41(DE3) cells (Lucigen) were transformed with a DrBphP-Avi-His or DrBphP-His expression construct and grown in 2×YT medium at 37° C. to an OD₆₀₀ of ~0.6 before induction with 0.1% arabinose at 25° C. for overnight. Harvested cell pellets from 1-liter cultures were resuspended in 40 mL ice-cold lysis buffer (50 mM sodium phosphate, pH 8.0, 300 mM NaCl, 10 mM imidazole, 10% glycerol) for sonication. The supernatant after centrifugation at 15,000×g, 4° C. for 30 min was loaded onto a 5 mL HisTrap™ column (GE Healthcare) pre-equilibrated with the lysis buffer. The column was washed with a washing buffer (50 mM sodium phosphate, pH 8.0, 300 mM NaCl, 20 mM imidazole, 10% glycerol) and then His-tagged DrBphP was eluted with an elution buffer (50 mM sodium phosphate, pH 8.0, 300 mM NaCl, 500 mM imidazole, 10% glycerol). Eluates were desalted with a HiPrep™ 26/10 column (GE Healthcare) pre-equilibrated with a storage buffer (1×PBS, 5% glycerol). Fractions were pooled and incubated with biliverdin (Frontier Scientific) with molar ratio of 1:20 at 4° C. overnight and then loaded onto a HiPrep™26/10 desalting column (GE Healthcare) pre-equilibrated with a storage buffer (1×PBS, 5% glycerol). Eluates were concentrated with Amicon Ultra-15 centrifugal filter units (30 kDa cutoff; Millipore). Concentrated proteins were loaded onto a HiLoad™ 16/600 Superdex 200 μg column (GE Healthcare) pre-equilibrated with a storage buffer (1×PBS, 5% glycerol). Eluted proteins were concentrated, examined by SDS-PAGE, and quantified by a Bradford assay (BioRad), then flash frozen by liquid N₂ and stored at −80° C.

His-tagged or Avi-Tagged nanobodies were expressed in Escherichia coli strain WK6 and purified by Ni-affinity and size-exclusion chromatography.

### Protein Biotinylation

DrBphP-Avi-His was biotinylated by BirA using a BirA-500 kit (Avidity). Typically, 200 μL BiomixA™ (10× concentration: 0.5 M bicine buffer, pH 8.3), 200 μL BiomixB (10× concentration: 100 mM ATP, 100 mM Mg(OAc)₂, 500 μM d-biotin), 200 μL BIO200 (10× concentration: 500 μM d-biotin), 20 μL 1 mg/mL BirA, and DrBphP-Avi-His (final concentration at ~2.4 mg/mL) were mixed with H₂O to a final volume of 2 mL. The biotinylation mixture was incubated at 37° C. for 1 h and then loaded onto a HiPrep™ 26/10 desalting column (GE Healthcare) pre-equilibrated with a storage buffer (1×PBS, 5% glycerol). Eluted proteins were concentrated, examined by SDS-PAGE, and quantified by the Bradford assay, flash frozen by liquid N₂, and stored at −80° C. LDB-3-Avi-His and LDB-14-Avi-His were biotinylated similarly as DrBphP.

### Phage Display Selection

The combinatorial nanobody phage library was prepared as previously described. Dimerization binders were selected using 775-nm and 654-nm illuminations for the negative and positive selections, respectively. Briefly, 1.2 mL 20 μM biotinylated DrBphP-Avi-His was bound to 600 μL streptavidin agarose resin (Thermo Scientific) and blocked with 1% casein and 1% BSA in 1×PBS pH 7.4 for 30 min at 4° C. in the dark. The resins were divided by a 2:1 ratio to pack the negative and positive selection columns (HR 5/5, GE Healthcare), respectively. As shown in FIG. 10, both columns were connected to AKTA FPLC system and equilibrated with ~10 mL PBS buffer at 0.5 mL/min. The negative and positive selection columns were illuminated with 775±14 nm light (FC-LED-780M, Prizmatix) at 0.8 mW/cm² for 10 min and 654±11 nm light (FC-LED-655A, Prizmatix) at 0.3 mW/cm² for 10 min and then wrapped with aluminum foil. The light intensity was measured with an optical power meter (PM100D, Thorlabs) connected to a S130C probe (Thorlabs). In each round, phage-displayed nanobodies were loaded onto the columns equilibrated with 1×PBS at a flow rate of 0.04 mL/min. Next, the negative selection column was removed and the positive selection column washed with ~30 mL 0.05% PBST (1×PBS with 0.05% v/v Tween 20) at a flow rate at 0.5 mL/min until the UV 280 nm baseline became stable (i.e., non-bound phages were washed out). Prior to the illumination, 2 mL flow through was collected as a “pre-elution fraction” at 0.5 mL/min immediately. The flow rate was decreased to 0 and then the positive selection column was illuminated with the 775-nm light (0.8 mW/cm²) for a given time (refer to FIG. 10). A 2-mL fraction was collected as a “light-elution fraction” at 0.5 mL/min immediately after the illumination. The percentage of phages specifically eluted by the light was estimated by comparing phage counts in the pre-elution and light-elution fractions. The light eluted phages were amplified and used as an input for next round biopanning.

### Y2H Screening

CDSs of the enriched nanobody library after four rounds of the biopanning were subcloned into pGADT7 to create a sub-library as preys. DrBphP was inserted to pGBKT7 as the bait. Y2HGold cells were co-transformed with bait and prey plasmids, plated onto SD/-Ade/-His/-Leu/-Trp plates under the 654-nm illumination (0.03 mW/cm²), and incubated at 30° C. for 4-5 days. ~2,000 well-grown clones were picked and grew in 1-mL SD/-Leu/-Trp medium in deep 96-well plates under the 654-nm illumination (0.03 mW/cm²) for 24 h. 1-μL cells of each clone were replica spotted to SD/-Ade/-His/-Leu/-Trp plates and incubated under the 654-nm illumination (0.03 mW/cm²) or in the dark for 2-3 days. Clones showing significantly faster growth under the illumination were picked for further analysis. Because clones picked from the plates were often contaminated with a small amount of other clones, plasmids were purified from yeast, transformed into an E. coli DH5α strain to select clones carrying pGADT7 on LB Agar plates with Ampicillin (100 μg/mL) and then identify those carrying correct nanobody genes by Sanger sequencing. To further confirm the gene activation specificity, sequenced preys and the bait were again co-transformed into Y2HGold cells; non-diluted and diluted (1/10 and 1/100) cells were spotted onto SD/-Ade/-His/-Leu/-Trp plates to compare colony growth under the illumination and in the dark. Sequence- and specificity-validated clones were chosen for further analyses.

### Phage ELISA

E. coli electrocompetent TG1 cells were transformed with pADL-23c inserted with selected nanobody candidates. Colonies were inoculated into 250 μL media (2×TY, 2% glucose, 100 μg/mL ampicillin) in deep 96-well plates and grown at 37° C. for overnight. 10-μL cultures were inoculated into 500 μL fresh media and cells were grown to OD₆₀₀=~0.5 and infected by CM13 helper phage with the multiplicity of infection (MOI) of ~18. Cells were shaken at 37° C. for 45 min, added with kanamycin (50 μg/mL, the final concentration), and grown at 25° C. for overnight. Plates were centrifuged for 30 min at 3,000×g and phage-containing supernatants were transferred to fresh plates for an ELISA assay. Specifically, ELISA plates (Nunc MaxiSorp™, Thermo Fisher Scientific) were coated with 100 μL 5 μg/mL streptavidin in a coating buffer (100 mM carbonate buffer, pH 8.6) at 4° C. for overnight. After washing five times with 0.05% PBST (1×PBS with 0.05% v/v Tween 20), each well was added with 100 μL 2 μM biotinylated DrBphP-Avi-His and incubated at room temperature (r.t.) for 1 h in the dark. Wells were washed five times with 0.05% PBST, blocked with 1% casein in 1×PBS, and then illuminated by the 654-nm (0.3 mW/cm²) or 775-nm (0.2 mW/cm²) light for 10 min. 100 μL phage supernatants were added and incubated at r.t. for 1 h in dark. Wells were washed 10 times with 0.05% PBST and then illuminated with corresponding light (654 nm at 0.3 mW/cm² or 775 nm at 0.2 mW/cm²) for 10 min before washing five times with 0.05% PBST. Wells were added with 100 μL HRP-M13 major coat protein antibody (RL-phl, Santa Cruz Biotechnology; 1:10,000 dilution with 1×PBS, 1% casein) and incubated at r.t. for 1 h in the dark. A colorimetric detection was performed using a 1-Step Ultra TMB ELISA substrate solution (Thermo Fisher Scientific); OD₄₅₀ was measured with a SpectraMax™ Plus 384 microplate reader (Molecular Devices).

### Mammalian Two-Hybrid Assay

HEK293T cells (ATCC, CRL-3216) were grown in a Dulbecco's modified Eagle's medium (DMEM) supplemented with 10% fetal bovine serum (FBS; Thermo Fisher Scientific) in a humidified incubator (Forma Scientific) under 5% CO₂ at 37° C. For a firefly luciferase assay, cells were grown in 24-well plates (Greiner Bio-One) to ~60% confluence and transiently co-transfected with the DrBphP bait and nanobody preys, and a luciferase reporter plasmid (Addgene, #64125) in a 1:1:1 ratio (0.25 μg each into a ~500 μL medium). After the transfection, culture medium was changed in 6 h and then cells were kept in the darkness for another 18 h prior to the transcription activation. The activation was performed by continuously illuminating cells with the 654-nm light at 0.2 mW/cm² for 24 h; cells were kept in the dark as the control. The time-course luciferase assay was performed as described above with different transcription induction times.

Luciferase levels were measured with a firefly luciferase glow assay kit (Pierce) following the manual. Briefly, after the transcription activation, cells were washed with 1×PBS, added with 150 μl of 1× cell lysis buffer, and incubated at 4° C. for 30 min. 20 μl cell lysate from each well was transferred into a black 96-well plate (CELLSTAR™, Greiner Bio-One, Cat #655079) and mixed with 50 μl of a Working Solution. Bioluminescence signals were measured with a SpectraMax™ i3 plate reader (Molecular Devices) after incubation in the dark at r.t. for 10 min.

The luciferase assay of RpBphP1-based systems was performed under the same condition, except that the transcription activation was performed with the 775-nm (0.2 mW/cm²) illumination, because different from DrBphP, RpBphP1 is converted to the light form by NIR illumination.

### Analysis of Nanobody Stability in Mammalian Cells

~2×10⁵ HEK293T cells were seeded in 6-well plates (Thermo Fisher Scientific, catalog #140675) in DMEM supplemented with 10% FBS, and incubated under 5% CO₂ at 37° C. for overnight. Cells in a 1.5-mL medium were transiently transfected with plasmids (2.5 μg each) encoding nanobody-SNAP-tag fusions using lipofectamine 2000 (Thermo Fisher Scientific). After 36-h incubation, the medium was removed and cells were washed with 1×PBS twice, dissociated from the plate by digestion with a 1× Trypsin-EDTA Solution (Thermo Fisher Scientific, catalog #R001100), and collected in 15-mL conical tubes. Cells were washed with 1 mL 1×PBS and re-suspended in 250 μL ice cold 1×PBS for sonication. After centrifugation at 20,000 g for 10 min, ~50 μL supernatants were incubated with 1 μM (final concentration) SNAP™-Surface 649 (New England Biolabs, catalog #S9159S) for 1 h at r.t. to label SNAP™-tagged proteins. Labelled samples were boiled for 10 min at 95° C. in an SDS sample loading buffer before loaded onto an SDS-PAGE gel. The gel was scanned by an Odyssey CLx™ imaging system (Li-cor Biosciences).

### Analytical SEC

Interactions of DrBphP with LDB-3 and LDB-14 were analyzed by analytical SEC. Samples were loaded onto a Superdex™ 200 Increase 10/300 GL column (GE Healthcare) pre-equilibrated with 1×PBS and eluted at 0.75 mL/min at 4° C. The column was calibrated with molecular weight standards (Bio-Rad, catalog #1511901). Light-sensitive samples were prepared in a dark room and the column and sample syringes were all covered by aluminum foil to avoid light exposure.

To detect the complex formation, DrBphP-His was photoconverted to the dark and light forms by the 775-nm (0.8 mW/cm², 10 min) and 654-nm (0.2 mW/cm², 5 min) illumination, respectively. ~6 μM (final concentration) DrBphP-His was added with ~5 μM (final concentration) LDB-3-His or LDB-14-His and incubated at r.t. for 30 min in the dark before loading a 500 μL mixture onto the column. 500-μL fractions with an elution volume between 8 and 16 mL were collected and proteins in each fraction were precipitated by trichloroacetic acid (TCA) for SDS-PAGE analysis. Briefly, 55 μL 100% TCA was mixed with each fraction and incubated at −20° C. for 30 min. After centrifugation at 20,000×g, 4° C. for 15 min, supernatants were removed, and pellets were washed with 600 μL ice-cold acetone three times and then dried in air. Pellets were resuspended and boiled in the SDS loading buffer and analyzed by SDS-PAGE.

### Isothermal Titration Calorimetry

Binding affinities and thermodynamics of LDB-3 and LDB-14 to DrBphP were measured by a MicroCal™ PEAQ-ITC device (Malvern) at 25° C. Specifically, ~210 μL of 10 or 5 μM DrBphP-His was loaded to a sample cell and then illuminated by the 654-nm (0.2 mW/cm², 5 min) or 775-nm (0.8 mW/cm², 15 min) light. ~38 μL 80 μM LDB-3-His and 50 μM LDB-14-His were titrated into 10 and 5 μM DrBphP-His in the cell, respectively, by 19 injections (2 μL each) from a syringe. Background heat transfer caused by the nanobody dilution was measured by conducting a titration of LDB-3-His (80 μM) or LDB-14-His (50 μM) into a 1×PBS buffer alone. Titration of 1×PBS buffer into DrBphP-His (10 μM) was also conducted as the control.

Raw ITC data were analyzed by NITPIC version 1.2.7. To find a suitable range for each injection, cut-off differentials for the injection end was changed to 0.1. The fitting equation for a one-site model is

$$y = \frac{L}{1 + {e}^{- k ( x - {x}_{0} )}} + b ,$$

where y represents the heat of injection, x represents the molar ratio, and b, k, L, x₀ are related parameters. The integrated data of LDB-3 and LDB-14 titrated to the light form were fitted with the above equation by using a “curve_fit” function in the Python-SciPy package, which generated K_{D}^{app} and other thermodynamic parameters in Table 4.

### Bio-Layer Interferometry

LDB-3 and LDB-14 binding kinetics were analyzed using an Octet™ RED96 system (ForteBio) and Streptavidin (SA) biosensors. Briefly, 20 μg/mL biotinylated LDB-3-Avi-His or LDB-14-Avi-His was immobilized on SA biosensors in 1×PBS buffer (pH 7.4). A duplicate set of sensors was incubated in the buffer without any protein to measure background binding. All sensors were blocked with a buffer (1×PBS, pH 7.4, 0.05% Tween-20, 0.2% BSA, and 10 ng/mL biocytin) before the binding assay. Serial dilutions of DrBphP-His in an assay buffer (1×PBS, pH 7.4, 0.05% Tween-20, and 0.2% BSA) were illuminated with the 654-nm (0.3 mW cm⁻², 5 min) or 775-nm (0.2 mW cm⁻², 10 min) light before binding to the nanobodies. The assay was performed in black 96-well plates with a total working volume of 0.2 mL per well at r.t. Raw data were analyzed by an Octet™ data analysis software V9.0 (ForteBio) using a double-reference-subtraction protocol to subtract signals related to nonspecific binding, background, and signal drift caused by sensor variability.

Apparent dissociation constants (K_{D}^{app}s) were calculated by the steady-state analysis and the fitting with a global 1:1 model. The fitting of apparent dissociation rate constant (k_{off}^{app}) was found to be more reliable (or less DrBphP-His concentration dependent) than the fitting of apparent binding rate constant (kₒₙ^{app}), so only k_{off}^{app} was calculated by fitting with the equation, C=C₀+A(1−e^{-k}^{_{off}}^{t}), where C represents the level of binding, C₀ the binding at the start of dissociation, A an asymptote, and t time. k_{off}^{app} for each binding was calculated using the “curve_fit” function in the Python-SciPy package. After obtaining K_{D}^{app} and k_{off}^{app}, kₒₙ^{app} was calculated by

$${k}_{on} = \frac{{k}_{off}}{{K}_{D}} .$$

Of note, compared with the fitting result, the dissociation curves were slightly tailed (FIG. 15), likely due to the contribution from DrBphP dimer dissociation.

### DrBphP Photoconversion Analysis

The DrBphP thermal relaxation efficiency was analyzed by absorption spectroscopy. Absorption spectra (500-900 nm) of DrBphP samples were obtained using a SpectraMax™ Plus 384 microplate reader (Molecular Devices). DrBphP-His was added in a quartz micro cuvette (Yixing Purshee Optical Elements) and then converted to the light or dark form by the 654-nm (0.5 mW/cm², 2 min) or 775-nm (0.3 mW/cm², 10 min) illumination before collecting spectra. To monitor the real-time thermal relaxation to the dark form, ~400 μL 5 μM (final concentration) DrBphP-His samples added with or without 5 μM (final concentration) LDB-3-His or LDB-14-His in the cuvette were first converted to the light form by the 654-nm (0.5 mW/cm²) illumination for 2 min and then immediately relaxed by the 775-nm (0.3 or 0.05 mW/cm²) illumination with different exposure times before collecting spectra. The ratio of A₇₅₀/A₇₀₀ was normalized to the range (0-1) to monitor the relaxation process.

### GFP Imaging

HEK293T cells were seeded in 10 cm Nunclon™ Delta Surface culture dishes (Thermo Scientific) in DMEM supplemented with 10% FBS in the humidified incubator under 5% CO₂ at 37° C. They were co-transfected with 10 μg a pGreenFire1-Gal4 lentivector (System Biosciences, catalog #TR017PA-1) and lentivirus-packing plasmids (5 μg each) including PMDL, REV and VSVG by a calcium phosphate transfection method. The medium was changed in 6 h after the transfection and the virus was harvested after incubation for another 72 h. To separate the virus from the medium, the medium was centrifuged at 500×g for 5 min and the supernatant was passing through a Millex-HV filter (0.45 μm, Merck Millipore). 2.5 out of 10 mL filtered virus was used to infect HEK293T cells cultured in another 10 cm dish under 50% confluence, with 10 μg/mL polybrene (Merck Millipore), for 24 h.

Lentivirus-transduced HEK293T cells were seeded in 35 mm glass bottom microwell dishes coated with poly-D-lysine (MatTek, catalog #P35GC-0-10-C) at a density of 1×10⁵ cells per dish. On the second day, cells were transiently co-transfected with the GAL4-BD-DrBphP and nanobody-p65 plasmids (1.25 μg each) using lipofectamine 2000 (Thermo Fisher Scientific) and incubated for overnight. For each nanobody candidate, two dishes were needed for the illumination and the dark control; after the transfection, dishes were immediately covered by aluminum foil to avoid light exposure. On the third day, cells were under the 654-nm (0.2 mW/cm²) illumination or maintained in the dark for another 48 h. Prior to fluorescence imaging, cells were fixed by 4% paraformaldehyde for 10 min and washed with 1×PBS.

GFP images were acquired using a Nikon Ti-E™ automated inverted microscope equipped with a Perfect Focus System, a Nikon 20×/0.75-NA Plan Apo Lambda objective, a linear encoded motorized stage (Nikon Ti-S-ER), and an Andor iXon Ultra 888 EMCCD camera (16-bit dynamic range, 1,024×1,024 array with 13-μm pixels). Cells were illuminated by a SPECTRA X™ LED illuminator (Lumencor) coupled with an excitation filter (448±19 nm) and a filter cube mounted with a dichroic mirror (506 nm) and an emission filter (510±20 nm) (Chroma). Culture dishes were scanned under the GFP and a brightfield channels. Acquired GFP images (dark and light condition) were analyzed by MATLAB for quantifying the fluorescence intensity. Specifically, fluorescence signals in all pixels were subtracted by an average background value (i.e., the median of the pixel intensity distribution in each field-of-view (FOV)) and integrated for each FOV. For each condition, 78 FOVs were sampled for statistical analysis.

### Thermodynamic Modeling of Competitive Hetero- and Homo-Dimerization

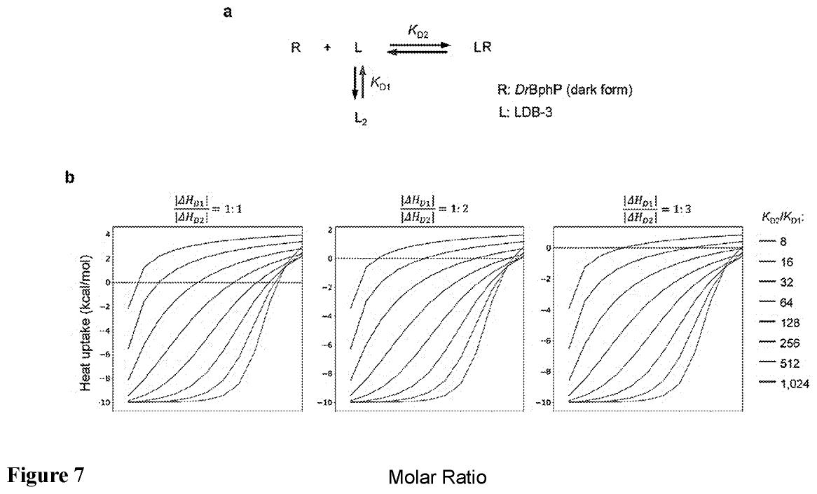

A simplified thermodynamic model was used to understand the observed transition of heat transfer from heat release to absorption when titrating LDB-3 to the dark-form DrBphP (FIG. 5a). We assume that the dark-form DrBphP-LDB-3 binding and LDB-3 dimerization are competitive (FIG. 7a): 2L↔L₂, and L+R↔LR, where L represents the monomeric LDB-3 and R the dark-form DrBphP. The dissociation constants are

$${K}_{D 1} = \frac{{[ L ]}^{2}}{[ {L}_{2} ]} = \frac{1}{{K}_{a 1}} and {K}_{D 2} = \frac{[ L ] [ R ]}{[ LR ]} = \frac{1}{{K}_{a 2}} ,$$

where [L], [L₂], [R], and [LR] are equilibrium concentrations, and Kₐ₁ and Kₐ₂ are association constants. The relationships of these concentrations are [L_{T}]=[L]+[LR]+2[L₂], and [R_{T}]=[R]+[LR], where [LT] represents the initial total concentration of LDB-3 and [R_{T}] represents the initial total concentration of DrBphP. So, the equilibrium dissociation constants can also be expressed as

$${K}_{D 1} = \frac{{( [ {L}_{T} ] - 2 [ {L}_{2} ] - [ LR ] )}^{2}}{[ {L}_{2} ]} ,$$

$$and$$

$${K}_{D 2} = \frac{( [ {L}_{T} ] - 2 [ {L}_{2} ] - [ LR ] ) ( [ {R}_{T} ] - [ LR ] )}{[ LR ]} .$$

[L₂] and [LR] could be determined if [K_{D1}], [K_{D2}], [L_{T}] and [R_{T}] are known.

The equilibrium dissociation constant is associated with the Gibbs energy of dissociation, ΔG_{D}, and can be expressed in terms of the enthalpy (ΔH_{D}) and entropy (ΔS_{D}) changes in the process: ΔG_{D}=−RT ln K_{D}=ΔH_{D}−TΔS_{D}. During the ITC assay, we assume that the whole heat transfer is the sum of ΔH_{D1} and ΔH_{D2} which could be calculated by concentration changes of each component using above equations. To simulate a titration process, the dissociation of the LDB-3 homodimer was set to be endothermic (ΔH_{D1}>0) while the formation of the LDB-3-DrBphP complex was exothermic (ΔH_{D2}<0), which is consistent with our experimental results (FIG. 5a and S7). To calculate heat transfer of the whole system, K_{D2}/K_{D1} was set as a variable, and

$$\frac{\vert Δ {H}_{D 1} \vert}{\vert Δ {H}_{D 2} \vert}$$

was set to be 1:1, 1:2, or 1:3. Thermographs were generated to show the integration of heat transfer in an titration experiment.

The simulation result showed that the clear transition from the heat release to absorption was found when KD2>>KD1 (e.g., KD2/KD1>100). The LDB-3 dimer is expected to a relatively weak complex because, in the SEC experiment, a large percentage of the dimer was dissociated at the low-μM concentrations (FIG. 13a). Based on our simulation and observed data (FIG. 5a), the dark-form DrBphP-LDB-3 complex (KD2) should be much weaker than the LDB-3 dimer (KD1), 3. It should be noted that this simplified model did not consider DrBphP dimerization and possible binding cooperativity in higher-order complexes. The fitting of the dark form binding data was found to be difficult due to the complexity of protein-protein interactions, so we did not calculate the KDapp. Nevertheless, the ITC experimental data and the modeling supports the low dark activity of LDB-3 observed in other assays.

### REFERENCES

(1) Xia, Y. Z.; Li, K.; Li, J. J.; Wang, T. Q.; Gu, L. C.; Xun, L. Y., T5 exonuclease-dependent assembly offers a low-cost method for efficient cloning and site-directed mutagenesis. Nucleic Acids Res. 2019, 47.
(2) Kang, S.; Davidsen, K.; Gomez-Castillo, L.; Jiang, H.; Fu, X.; Li, Z.; Liang, Y.; Jahn, M.; Moussa, M.; DiMaio, F.; Gu, L., COMBINES-CID: An efficient method for de novo engineering of highly specific chemically induced protein dimerization systems. J. Am. Chem. Soc. 2019, 141, 10948-10952.
(3) Scheuermann, T. H.; Brautigam, C. A., High-precision, automated integration of multiple isothermal titration calorimetric thermograms: New features of NITPIC. Methods 2015, 76, 87-98.
(4) Takala, H.; Bjorling, A.; Berntsson, O.; Lehtivuori, H.; Niebling, S.; Hoemke, M.; Kosheleva, I.; Henning, R.; Menzel, A.; Ihalainen, J. A.; Westenhoff, S., Signal amplification and transduction in phytochrome photosensors. Nature 2014, 509, 245-248.
(5) Kliebenstein, D. J.; Lim, J. E.; Landry, L. G.; Last, R. L., Arabidopsis UVR8 regulates ultraviolet-B signal transduction and tolerance and contains sequence similarity to human Regulator of Chromatin Condensation 1. Plant Physiol. 2002, 130, 234-243.
(6) Wu, D.; Hu, Q.; Yan, Z.; Chen, W.; Yan, C. Y.; Huang, X.; Zhang, J.; Yang, P. Y.; Deng, H. T.; Wang, J. W.; Deng, X. W.; Shi, Y. G., Structural basis of ultraviolet-B perception by UVR8. Nature 2012, 484, 214-U96.
(7) Chen, D.; Gibson, E. S.; Kennedy, M. J., A light-triggered protein secretion system. J. Cell Biol. 2013, 201, 631-640.
(8) Hirose, Y.; Shimada, T.; Narikawa, R.; Katayama, M.; Ikeuchi, M., Cyanobacteriochrome CcaS is the green light receptor that induces the expression of phycobilisome linker protein. Proc. Natl. Acad. Sci. U.S.A 2008, 105, 9528-9533.
(9) Abe, K.; Miyake, K.; Nakamura, M.; Kojima, K.; Ferri, S.; Ikebukuro, K.; Sode, K., Engineering of a green-light inducible gene expression system in Synechocystis sp PCC6803. Microb. Biotechnol. 2014, 7, 177-183.
(10) Blain-Hartung, M.; Rockwell, N. C.; Moreno, M. V.; Martin, S. S.; Gan, F.; Bryant, D. A.; Lagarias, J. C., Cyanobacteriochrome-based photoswitchable adenylyl cyclases (cPACs) for broad spectrum light regulation of cAMP levels in cells. J. Biol. Chem. 2018, 293, 8473-8483.
(11) Song, J. Y.; Cho, H. S.; Cho, J. I.; Jeon, J. S.; Lagarias, J. C.; Park, Y. I., Near-UV cyanobacteriochrome signaling system elicits negative phototaxis in the cyanobacterium Synechocystis sp PCC 6803. Proc. Natl. Acad. Sci. U.S.A 2011, 108, 10780-10785.
(12) Ramakrishnan, P.; Tabor, J. J., Repurposing synechocystis PCC6803 UirS-UirR as a UV-violet/green photoreversible transcriptional regulatory tool in E. coli. ACS Synth. Biol. 2016, 5, 733-740.
(13) Okajima, K.; Yoshihara, S.; Fukushima, Y.; Geng, X. X.; Katayama, M.; Higashi, S.; Watanabe, M.; Sato, S.; Tabata, S.; Shibata, Y.; Itoh, S.; Ikeuchi, M., Biochemical and functional characterization of BLUF-type flavin-binding proteins of two species of cyanobacteria. J. Biochem. 2005, 137, 741-750.
(14) Yuan, H.; Dragnea, V.; Wu, Q.; Gardner, K. H.; Bauer, C. E., Mutational and structural studies of the PixD BLUF output signal that affects light-regulated interactions with PixE. Biochemistry 2011, 50, 6365-6375.
(15) Masuda, S.; Nakatani, Y.; Ren, S. K.; Tanaka, M., Blue light-mediated manipulation of transcription factor activity in vivo. ACS Chem. Biol. 2013, 8, 2649-2653.
(16) Stierl, M.; Stumpf, P.; Udwari, D.; Gueta, R.; Hagedorn, R.; Losi, A.; Gartner, W.; Petereit, L.; Efetova, M.; Schwarzel, M.; Oertner, T. G.; Nagel, G.; Hegemann, P., Light modulation of cellular cAMP by a small bacterial photoactivated adenylyl cyclase, bPAC, of the soil bacterium Beggiatoa. J. Biol. Chem. 2011, 286, 1181-1188.
(17) Lindner, R.; Hartmann, E.; Tarnawski, M.; Winkler, A.; Frey, D.; Reinstein, J.; Meinhart, A.; Schlichting, I., Photoactivation mechanism of a bacterial light-regulated adenylyl cyclase. J. Mol. Biol. 2017, 429, 1336-1351.
(18) Ryu, M. H.; Moskvin, O. V.; Siltberg-Liberles, J.; Gomelsky, M., Natural and engineered photoactivated nucleotidyl cyclases for optogenetic applications. J. Biol. Chem. 2010, 285, 41501-41508.
(19) Salomon, M.; Christie, J. M.; Knieb, E.; Lempert, U.; Briggs, W. R., Photochemical and mutational analysis of the FMN-binding domains of the plant blue light receptor, phototropin. Biochemistry 2000, 39, 9401-9410.
(20) Halavaty, A. S.; Moffat, K., N- and C-terminal flanking regions modulate light-induced signal transduction in the LOV2 domain of the blue light sensor phototropin 1 from Avena sativa. Biochemistry 2007, 46, 14001-14009.
(21) Strickland, D.; Moffat, K.; Sosnick, T. R., Light-activated DNA binding in a designed allosteric protein. Proc. Natl. Acad. Sci. U.S.A 2008, 105, 10709-10714.
(22) Losi, A.; Polverini, E.; Quest, B.; Gartner, W., First evidence for phototropin-related blue-light receptors in prokaryotes. Biophys. J. 2002, 82, 2627-2634.
(23) Moglich, A.; Moffat, K., Structural basis for light-dependent signaling in the dimeric LOV domain of the photosensor YtvA. J. Mol. Biol. 2007, 373, 112-126.
(24) Moglich, A.; Ayers, R. A.; Moffat, K., Design and signaling mechanism of light-regulated histidine kinases. J. Mol. Biol. 2009, 385, 1433-1444.
(25) Heintzen, C.; Loros, J. J.; Dunlap, J. C., The PAS protein VIVID defines a clock-associated feedback loop that represses light input, modulates gating, and regulates clock resetting. Cell 2001, 104, 453-464.
(26) Zoltowski, B. D.; Schwerdtfeger, C.; Widom, J.; Loros, J. J.; Bilwes, A. M.; Dunlap, J. C.; Crane, B. R., Conformational switching in the fungal light sensor vivid. Science 2007, 316, 1054-1057.
(27) Wang, X.; Chen, X. J.; Yang, Y., Spatiotemporal control of gene expression by a light-switchable transgene system. Nat. Methods 2012, 9, 266-U64.
(28) Nelson, D. C.; Lasswell, J.; Rogg, L. E.; Cohen, M. A.; Bartel, B., FKF1, a clock-controlled gene that regulates the transition to flowering in Arabidopsis. Cell 2000, 101, 331-340.
(29) Nakasako, M.; Zikihara, K.; Matsuoka, D.; Katsura, H.; Tokutomi, S., Structural basis of the LOV1 dimerization of Arabidopsis phototropins 1 and 2. J. Mol. Biol. 2008, 381, 718-733.
(30) Yazawa, M.; Sadaghiani, A. M.; Hsueh, B.; Dolmetsch, R. E., Induction of protein-protein interactions in live cells using light. Nat. Biotechnol. 2009, 27, 941-U105.
(31) Nash, A. I.; McNulty, R.; Shillito, M. E.; Swartz, T. E.; Bogomolni, R. A.; Luecke, H.; Gardner, K. H., Structural basis of photosensitivity in a bacterial light-oxygen-voltage/helix-turn-helix (LOV-HTH) DNA-binding protein. Proc. Natl. Acad. Sci. U.S.A 2011, 108, 9449-9454.
(32) Motta-Mena, L. B.; Reade, A.; Mallory, M. J.; Glantz, S.; Weiner, O. D.; Lynch, K. W.; Gardner, K. H., An optogenetic gene expression system with rapid activation and deactivation kinetics. Nat. Chem. Biol. 2014, 10, 196-202.
(33) Guo, H. W.; Yang, W. Y.; Mockler, T. C.; Lin, C. T., Regulations of flowering time by Arabidopsis photoreceptors. Science 1998, 279, 1360-1363.
(34) Kennedy, M. J.; Hughes, R. M.; Peteya, L. A.; Schwartz, J. W.; Ehlers, M. D.; Tucker, C. L., Rapid blue-light-mediated induction of protein interactions in living cells. Nat. Methods 2010, 7, 973-U48.
(35) Zhou, X. X.; Chung, H. K.; Lam, A. J.; Lin, M. Z., Optical control of protein activity by Ffuorescent protein domains. Science 2012, 338, 810-814.
(36) Mizuno, H.; Mal, T. K.; Walchli, M.; Kikuchi, A.; Fukano, T.; Ando, R.; Jeyakanthan, J.; Taka, J.; Shiro, Y.; Ikura, M.; Miyawaki, A., Light-dependent regulation of structural flexibility in a photochromic fluorescent protein. Proc. Natl. Acad. Sci. U.S.A 2008, 105, 9227-9232.
(37) Baca, M.; Borgstahl, G. E. O.; Boissinot, M.; Burke, P. M.; Williams, D. R.; Slater, K. A.; Getzoff, E. D., Complete chemical structure of photoactive yellow protein: novel thioester-linked 4-hydroxycinnamyl chromophore and photocycle chemistry. Biochemistry 1994, 33, 14369-14377.
(38) Ali, A. M.; Reis, J. M.; Xia, Y.; Rashid, A. J.; Mercaldo, V.; Walters, B. J.; Brechun, K. E.; Borisenko, V.; Josselyn, S. A.; Karanicolas, J.; Woolley, G. A., Optogenetic inhibitor of the transcription factor CREB. Chem. Biol. 2015, 22, 1531-1539.
(39) Scheib, U.; Stehfest, K.; Gee, C. E.; Korschen, H. G.; Fudim, R.; Oertner, T. G.; Hegemann, P., The rhodopsin-guanylyl cyclase of the aquatic fungus Blastocladiella emersonii enables fast optical control of cGMP signaling. Sci. Signal. 2015, 8.
(40) Kainrath, S.; Stadler, M.; Reichhart, E.; Distel, M.; Janovjak, H., Green-light-induced inactivation of receptor signaling using cobalamin-binding domains. Angew. Chem. Int. Ed. Engl. 2017, 56, 4608-4611.
(41) Kaberniuk, A. A.; Shemetov, A. A.; Verkhusha, V. V., A bacterial phytochrome-based optogenetic system controllable with near-infrared light. Nat. Methods 2016, 13, 591-597.
(42) Bellini, D.; Papiz, M. Z., Structure of a bacteriophytochrome and light-stimulated protomer swapping with a gene repressor. Structure 2012, 20, 1436-1446.
(43) Davis, S. J.; Vener, A. V.; Vierstra, R. D., Bacteriophytochromes: Phytochrome-like photoreceptors from nonphotosynthetic eubacteria. Science 1999, 286, 2517-2520.
(44) Shu, X. K.; Royant, A.; Lin, M. Z.; Aguilera, T. A.; Lev-Ram, V.; Steinbach, P. A.; Tsien, R. Y., Mammalian expression of infrared fluorescent proteins engineered from a bacterial phytochrome. Science 2009, 324, 804-807.
(45) Hughes, J.; Lamparter, T.; Mittmann, F.; Hartmann, E.; Gartner, W.; Wilde, A.; Borner, T., A prokaryotic phytochrome. Nature 1997, 386, 663-663.
(46) Essen, L. O.; Mailliet, J.; Hughes, J., The structure of a complete phytochrome sensory module in the Pr ground state. Proc. Natl. Acad. Sci. U.S.A 2008, 105, 14709-14714.
(47) Levskaya, A.; Chevalier, A. A.; Tabor, J. J.; Simpson, Z. B.; Lavery, L. A.; Levy, M.; Davidson, E. A.; Scouras, A.; Ellington, A. D.; Marcotte, E. M.; Voigt, C. A., Engineering Escherichia coli to see light—These smart bacteria ‘photograph’ a light pattern as a high-definition chemical image. Nature 2005, 438, 441-442.
(48) Sharrock, R. A.; Quail, P. H., Novel phytochrome sequences in Arabidopsis thaliana: structure, evolution, and differential expression of a plant regulatory photoreceptor family. Genes Dev. 1989, 3, 1745-1757.
(49) Burgie, E. S.; Bussell, A. N.; Walker, J. M.; Dubiel, K.; Vierstra, R. D., Crystal structure of the photosensing module from a red/far-red light-absorbing plant phytochrome. Proc. Natl. Acad. Sci. U.S.A. 2014, 111, 10179-10184.
(50) Shimizu-Sato, S.; Huq, E.; Tepperman, J. M.; Quail, P. H., A light-switchable gene promoter system. Nat. Biotechnol. 2002, 20, 1041-1044.

We claim:

1. A kit, comprising ore or more of:
(I) a Deinococcus radiodurans bacteriophytochrome (DrBphP) light form-binding single chain antibody, wherein single chain antibody comprises a set of complementarity-determining regions (CDRs) selected from the group consisting of:
(a) SEQ ID NO:1 (CDR1), SEQ ID NO:2 (CDR2), and SEQ ID NO:3 (CDR3); or
(b) SEQ ID NO:4 (CDR1), SEQ ID NO:5 (CDR2), and SEQ ID NO:6 (CDR3); or
(II) a nucleic acid encoding a Deinococcus radiodurans bacteriophytochrome (DrBphP) light form-binding single chain antibody, wherein single chain antibody comprises a set of complementarity-determining regions (CDRs) selected from the group consisting of:
(a) SEQ ID NO:1 (CDR1), SEQ ID NO:2 (CDR2), and SEQ ID NO:3 (CDR3); or
(b) SEQ ID NO:4 (CDR1), SEQ ID NO:5 (CDR2), and SEQ ID NO:6 (CDR3); or
(III) an expression vector comprising a nucleic acid encoding a Deinococcus radiodurans bacteriophytochrome (DrBphP) light form-binding single chain antibody, wherein single chain antibody comprises a set of complementarity-determining regions (CDRs) selected from the group consisting of:
(a) SEQ ID NO:1 (CDR1), SEQ ID NO:2 (CDR2), and SEQ ID NO:3 (CDR3); or
(b) SEQ ID NO:4 (CDR1), SEQ ID NO:5 (CDR2), and SEQ ID NO:6 (CDR3); or
(IV) a cell that comprising an expression vector, wherein the expression vector comprises a nucleic acid encoding a Deinococcus radiodurans bacteriophytochrome (DrBphP) light form-binding single chain antibody, wherein single chain antibody comprises a set of complementarity-determining regions (CDRs) selected from the group consisting of:
(a) SEQ ID NO:1 (CDR1), SEQ ID NO:2 (CDR2), and SEQ ID NO:3 (CDR3); or
(b) SEQ ID NO:4 (CDR1), SEQ ID NO:5 (CDR2), and SEQ ID NO:6 (CDR3).

2. The kit of claim 1, comprising a Deinococcus radiodurans bacteriophytochrome (DrBphP) light form-binding single chain antibody, wherein single chain antibody comprises a set of complementarity-determining regions (CDRs) selected from the group consisting of:
(a) SEQ ID NO:1 (CDR1), SEQ ID NO:2 (CDR2), and SEQ ID NO:3 (CDR3); or
(b) SEQ ID NO:4 (CDR1), SEQ ID NO:5 (CDR2), and SEQ ID NO:6 (CDR3); wherein the single chain antibody comprises an amino acid sequence at least 40% identical to the amino acid sequence of SEQ ID NO:9 or SEQ ID NO:10.

3. The kit of claim 2, wherein the single chain antibody comprises an amino acid sequence at least 60% identical to the amino acid sequence of SEQ ID NO:9 or SEQ ID NO:10.

4. The kit of claim 2, wherein the single chain antibody comprises an amino acid sequence at least 90% identical to the amino acid sequence of SEQ ID NO:9 or SEQ ID NO:10.

5. The kit of claim 2, wherein the single chain antibody comprises an amino acid sequence at least 80% identical to the amino acid sequence of SEQ ID NO:9.

6. The kit of claim 2, wherein the single chain antibody comprises an amino acid sequence at least 80% identical to the amino acid sequence of SEQ ID NO:10.

7. The kit of claim 2, wherein the single chain antibody comprises an amino acid sequence at least 90% identical to the amino acid sequence of SEQ ID NO:9.

8. The kit of claim 2, wherein the single chain antibody comprises an amino acid sequence at least 90% identical to the amino acid sequence of SEQ ID NO:10.

9. The kit of claim 2, wherein the single chain antibody comprises the amino acid sequence of SEQ ID NO:9.

10. The kit of claim 2, wherein the single chain antibody comprises the amino acid sequence of SEQ ID NO:9.

11. The kit of claim 1, further comprising one or more of:
(I) a photosensory module of Deinococcus radiodurans bacteriophytochrome (DrBphP) comprising a polypeptide at least 70% identical to the amino acid sequence of SEQ ID NO: 11; or
(II) a nucleic acid encoding a photosensory module of Deinococcus radiodurans bacteriophytochrome (DrBphP) comprising a polypeptide at least 70% identical to the amino acid sequence of SEQ ID NO: 11; or
(III) an expression vector comprising a nucleic acid encoding a photosensory module of Deinococcus radiodurans bacteriophytochrome (DrBphP) comprising a polypeptide at least 70% identical to the amino acid sequence of SEQ ID NO: 11; or
(IV) a cell comprising an expression vector comprising a nucleic acid encoding a photosensory module of Deinococcus radiodurans bacteriophytochrome (DrBphP) comprising a polypeptide at least 70% identical to the amino acid sequence of SEQ ID NO: 11.

12. The kit of claim 11, comprising a photosensory module of Deinococcus radiodurans bacteriophytochrome (DrBphP) comprising a polypeptide at least 70% identical to the amino acid sequence of SEQ ID NO: 11.

13. The kit of claim 12, wherein the photosensory module of Deinococcus radiodurans bacteriophytochrome (DrBphP) comprises a polypeptide at least 80% identical to the amino acid sequence of SEQ ID NO: 11.

14. The kit of claim 12, wherein the photosensory module of Deinococcus radiodurans bacteriophytochrome (DrBphP) comprises a polypeptide at least 90% identical to the amino acid sequence of SEQ ID NO: 11.

15. The kit of claim 12, wherein the photosensory module of Deinococcus radiodurans bacteriophytochrome (DrBphP) comprises the amino acid sequence of SEQ ID NO: 11.
